(19) United States
(12) Patent Application Publication
(10) Pub. No.: US 2023/0240601 A1
(43) Pub. Date: Aug. 3, 2023

(54) METHOD FOR IMPROVING BEHAVIORAL DEFICITS OF SUBJECT AT RISK FOR OR IN EARLY STAGE OF ALZHEIMER DISEASE OR OTHER NEURODEGENERATIVE DISEASES

(71) Applicant: Naval Medical Research Center, Silver Spring, MD (US)

(72) Inventors: Stephen T. Ahlers, Olney, MD (US); Anna E. Tschiffely, Walkersville, MD (US)

(73) Assignee: The United States of America as represented by the secretary of the Navy, Silver Spring, MD (US)

(21) Appl. No.: 18/105,059

(22) Filed: Feb. 2, 2023

Related U.S. Application Data
(60) Provisional application No. 63/305,748, filed on Feb. 2, 2022.

(51) Int. Cl. A61B 5/00; G01N 33/68; A61K 45/06

(52) U.S. Cl. CPC A61B 5/4884; A61B 5/4064; G01N 33/6803; A61K 45/06

(57) ABSTRACT

This invention discloses a method for improving behavioral deficits and lowering brain Aβ42 in subjects at risk for or in early stage of Alzheimer's disease or other neurodegenerative diseases by exposing said subject to frequent repetitive low intensity blast overpressure. The invention also discloses a method for treating and preventing Alzheimer's disease or other neurodegenerative diseases and associated conditions by reduce abnormal accumulation of brain protein, improve brain inflammation and clearance of amyloid beta.

### CROSS-REFERENCE TO RELATED APPLICATIONS

This application claims priority to U.S. Provisional Application No. 63/305,748 filed on Feb. 2, 2022, which is hereby incorporated by reference.

### FEDERALLY SPONSORED RESEARCH AND DEVELOPMENT

This invention was made with government support under was supported by the Department of Veterans Affairs, Veterans Health Administration, Rehabilitation Research and Development Service Awards 1I01RX002660 (GE), 1I01RX000684 (SG), and 1I01RX002333 (SG), 1I21RX003459 (MAGS) and 121RX002876 (MAGS), the Department of Veterans Affairs Office of Research and Development Medical Research Service 1I01BX004067 (GE) and 1I01BX002311 (DC), by Defense Health Program (DHP) work unit number 603115HP.3520.001.A1411 from Joint Program Committee 5 (STA), the Alzheimer's Drug Discovery Foundation (SG) and by NIA P50 AG005138 and P30 AG066514 both to Mary Sano (SG, PRH). The government has certain rights in the invention.

### FIELD OF THE INVENTION

The present invention relates generally to the treatment and prevention of Alzheimer's disease or other neurodegenerative diseases, and specifically to a method for treating and improving behavioral deficits in patients at risk or in early stage of Alzheimer's disease (Aβ) like or other neurodegenerative diseases.

### PRIOR ART

Aspects of this invention was previously disclosed in Perez Garcia G, De Gasperi R, Tschiffely A E, Gama Sosa M A, Abutarboush R, Kawoos U, Statz J K, Ciarlone S, Reed E, Jeyarajah T, Perez G M, Otero-Pagan A, Pryor D, Hof P R, Cook D G, Gandy S, Elder G A, Ahlers S T (2021) Repetitive Low-Level Blast Exposure Improves Behavioral Deficits and Chronically Lowers A042 in an Alzheimer Disease Transgenic Mouse Model. J Neurotrauma 38:3146-3173, by the inventor or a joint inventor and De Gasperi R, Gama Sosa M A, Kim S H, Steele J W, Shaughness M C, Maudlin-Jeronimo E, Hall A A, Dekosky S T, McCarron R M, Nambiar M P, Gandy S, Ahlers S T, Elder G A (2012) Acute blast injury reduces brain abeta in two rodent species. Front Neurol 3:177. Both references are hereby incorporated by reference.

### BACKGROUND OF THE INVENTION

The long-term effects of exposure to primary blast overpressure (BOP) are an important health concern in military personnel. Blast-related neurotrauma remains the leading cause of injury in deployed United States military personnel accounting for up to a third of all treated traumatic brain injuries (TBI). Exposure to blast resulting from proximity to explosive blasts is common in military personnel, occurring mostly during non-combat training exercises, and may occasionally be associated with mild TBI symptoms. However, seemingly innocuous subclinical “low-intensity” acute and repetitive blast exposures devoid of acute neurological effects and a TBI diagnosis at the time of exposure may nevertheless lead to long-term changes in the brain, including neuropathology and an increased risk for neurodegenerative diseases later in life.

Although numerous studies examined blast-related changes in the brain in human subjects and in preclinical models, the effects of low-intensity BOP on the brain are yet to be fully understood. Both human and animal studies have documented that non-blast TBI may lead to pathological changes of extracellular and intracellular proteins associated with neurodegenerative diseases, including amyloid beta protein (Aβ) and tau. Amyloid plaques and hyperphosphorylated tau neurofibrillary tangles are the two cardinal histological features of Alzheimer's Disease (Aβ), while tau pathological changes are characteristic of chronic traumatic encephalopathy (CTE). In AD, the amyloid load manifesting as Aβ deposits in blood vessels (congophylic amyloid angiopathy (CAA)) and plaques in the brain parenchyma are believed to lead to neuronal death and the ensuing dementia. This hypothesis has been challenged by evidence that smaller oligomeric Aβ peptides may be more harmful than plaques and are associated with memory impairment.

Some studies linked moderate and severe non-blast TBI with loss of consciousness to AD as a precipitating or accelerating factor, while other studies failed to find a clear association between the future risk of AD and TBI-induced changes in Aβ peptides. Although the disease process leading to cognitive impairment in AD and TBI may very well be different, there are some notable similarities involving changes in Aβ production, clearance, and dysregulation of the cerebral vasculature, resulting in alterations in the levels of Aβ peptides. A clinical study showed elevated Aβ as early as 2 hours after a single non-blast TBI event in the human cortex. The pathophysiology of blast-related TBI is less clear than that of non-blast TBI; however, there is some evidence that exposure to blast also alters Aβ biology in the brain. Elevation in serum levels of Aβ peptides have been reported in military service members. In preclinical models, exposure to subclinical low-intensity blast was associated with an unexpected reduction in Aβ peptides in rodent models in the acute phase after blast exposure (De Gasperi et al., 2012). This reduction in Aβ was not observed after exposure to high intensity blasts. The inventors replicated these findings in an APP/PSI transgenic AD model exposed to low-intensity blast and documented reductions in Aβ40 and 42 (Perez Garcia et al., 2021). APP/PSI are double transgenic mice expressing a chimeric mouse/human amyloid precursor protein (Mo/HuAPP695swe) and a mutant human presenilin 1 (PS1-dE9), both directed to CNS neurons. Both mutations are associated with early-onset Alzheimer's disease. These mice may be useful in studying neurological disorders of the brain, specifically Alzheimer's disease, amyloid plaque formation and aging. These findings suggest that non-blast TBI and blast-related TBI have distinct effects on Aβ in the brain and that the intensity of blast overpressure is a critical parameter in determining the ensuing alterations in the brain.

Alterations in the levels of Aβ in the brain can be related to an imbalance in the production or clearance and degradation of the protein. Excessive production of amyloid precursor protein (APP) or Aβ peptides or diminished clearance of Aβ may enhance its accumulation. APP is synthesized in neuronal soma, transported to axons, and rapidly cleaved by proteolytic enzymes in two alternative pathways: an amyloidogenic (Aβ-producing) and a non-amyloidogenic pathway. The major Aβ-producing pathway involves endosomal cleavage of APP by β-secretase (β-site APP cleaving enzyme; BACE1) followed by γ-secretase (presenlin1; PSN1), releasing Aβ into the synaptic cleft (O'Brien and Wong, 2011). In the non-amyloidogenic pathway, proteins with α-secretase activity, cleave APP releasing sAPPα, the N-terminal domain of APP. These proteins with α-secretase activity include those in the disintegrin and metalloproteinase (ADAM) family, ADAM10 and ADAM17.

Accumulation of APP has been documented in injured axons and increase in APP levels is often used to demonstrate axonal injury. APP levels increase after severe TBI in human cases and animal models. Increase in both β and γ-secretase components have been reported after non-blast TBI in humans and in animal experimental models. Conversely, enhancement of APP processing in the non-amyloidogenic pathway may attenuate Aβ accumulation, provide neuroprotection, and prevent amyloid-induced vascular pathology in AD.

Deficiencies in enzymatic degradation or clearance of Aβ are implicated in enhancing accumulation and aggregation of Aβ in neurotoxic oligomers and plaques. Routes of Aβ clearance from the brain include: proteolytic degradation of the peptide by proteases expressed by glial, endothelial, and other cell types; transvascular transportation across the blood-brain barrier (BBB); “glymphatic” clearance through aquaporin-4 (AQP4) water channels in astrocytes; and perivascular (specifically, periarterial) flow and drainage of solutes in cerebrospinal fluid (CSF) through cervical lymph nodes possibly via meningeal lymphatic vessels.

The cerebral vasculature plays a significant role in the clearance of AB and other solutes in the brain. The majority of Aβ is removed from the brain by transcytosis across endothelial cells by low density lipoprotein receptor-related protein-1 (LRP-1). Dysregulation of the LRP-1 receptor at the BBB is associated with accumulation of Aβ in animal models and in human AD cases. Bulk flow of interstitial fluid (ISF) via the glymphatic system mediates exchange of solutes and waste products in ISF into CSF. The system relies on the arteriolar pulsatility and AQP4 channels and may be most active during sleep. Changes in the density and distribution of AQP4 have been associated with aggregation misfolded proteins, including Aβ, and neurodegenerative changes in the brain. This invention provides a new method that is used for the treatment and prevention of Alzheimer's disease (AD) or other neurodegenerative diseases and to improve associated behavioral deficits of subjects at risk for or in early stage of AD or other neurodegenerative diseases by administering to the subject repetitive low intensity blast overpressure.

### SUMMARY OF THE INVENTION

The inventor has identified molecular pathways that associated with AD and other neurodegenerative diseases and exploited them in the new methods to improve the therapy and prevention of AD and other neurodegenerative diseases and associated conditions. Some embodiments of the present invention provide a system and methods for treating Alzheimer's disease.

For some applications, a method for treating Alzheimer's and other neurodegenerative diseases and associated conditions comprises: 1) Identifying a subject at risk of developing or in early stages of Alzheimer's disease or other neurodegenerative diseases; and 2) exposing said subject to frequent repetitive low intensity blast overpressure.

For some applications, treating the subject comprises facilitating clearance of amyloid beta by improving LRP1-mediated transcytosis through the endothelium and/or altering AQP4-aided glymphatic clearance. Therefore, the invention provides new methods for altering enzymatic, transvascular, and perivascular clearance of Aβ using repetitive frequent low intensity blast overpressure.

### BRIEF DESCRIPTION OF DRAWINGS

FIG. 1. Repetitive low-level blast exposure in an Alzheimer's disease transgenic mouse model: Timeline of experiments for cohorts 1 and 2

FIG. 2. Histopathology in APP/PS1 Tg mice after blast exposure. Nissl staining in the hippocampus and neocortex (A, B) and cerebellum (C, D) is shown from sham- (A, C) and blast-exposed (B, D) APP/PS1 Tg mice sacrificed at 7 weeks after the last blast exposure (35 weeks of age). No significant histological changes were noted. Scale bar=200 [symbol] m

FIG. 3. Elevated zero maze (EZM), light dark (LD) and open-field testing of cohort 1. APP/PS1 Tg mice were exposed to blast (n=7) or sham (n=8) conditions beginning at 20 weeks of age and received three blast exposures per week for 8 weeks. Behavioral testing was begun at 30 weeks of age (FIG. 1). For the EZM (A), time in motion (Move Time), mean speed, open arm entries, open arm time and the latency to cross into the second open arm (Cross Arm Latency) area shown. In the LD task (B), the latency to the light edge, latency to reach the light center, entries into the light center, as a well as time total time spent on the light side and total distance traveled on the light side are shown. For the open field (C) time in motion (Move Time), total distance traveled, the latency to the open field center, center entries and time spent in the center of the open field are shown. Error bars indicate the standard error of the mean (SEM). Asterisks indicate values significantly different between groups (*p<0.05, **p<0.01, unpaired t-tests).

FIG. 4. Novel object recognition (NOR) testing of cohort 1. Blast-exposed (n=7) and sham-exposed (n=8) APP/PS1 transgenic (Tg) mice from cohort 1 were tested in novel object recognition (NOR) and novel object localization (NOL) tasks. Panel (A) shows time spent exploring the objects (OB1 and OB2) during the NOR training session as well as exploration of the previously presented familiar object (FO) compared to the novel object (NO) when presented 1 h (short-term memory, STM) or 24 h (long-term memory, LTM) later. Panels (B) and (C) show the discrimination index (B) and total time spend exploring the objects (C) during the indicated NOR sessions. Panel (D) shows time spent exploring the objects (OB1 and OB2) during the NOL training session as well as exploration of the previously presented objects in their familiar location (FL) compared to a novel location (NL) when presented 1 h later (short-term memory, STM). Asterisks indicate values significantly different between groups (*p<0.05, ***p<0.001, unpaired t-tests).

FIG. 5. Testing of cohort 1 in the Barnes maze and fear learning. Blast-exposed (n=7) and control (n=8) mice from cohort 1 were tested in a Barnes maze or fear conditioning paradigm. For the Barnes maze total distance moved, time to enter the target quadrant and time to enter the escape hole are shown across the five trials. A repeated measures ANOVA revealed a significant within subjects effect by trial (F_{2.069, 26.902}=5.973, p=0.007) for distance moved but no effect of trial*condition (F_{2.069, 26.902}=1.211, p=0.315). However, a test of between subject effects revealed a significant group difference with the Tg Blast moving more (F_{1, 13}=6.976, p=0.020). A repeated measures ANOVA of the time to first enter the target quadrant revealed no significant within subjects effect by trial (F_{2.180, 28.399}=0.906, p=0.467) or effect of trial*condition (F_{2.180, 28.399}=0.230, p=0.814). However, a test of between subject effects revealed a significant group difference with the Tg Blast exhibiting shorter latencies (F₁₃=8.973, p=0.010). A repeated measures ANOVA of the time to enter the target revealed a significant within subjects effect by trial (F_{4, 52}=13.503, p<0.001) but no effect of trial*condition (F_{4, 52}=0.108, p=0.979). A test of between subject effects again revealed a significant group difference with the Tg Blast exhibiting shorter latencies (F_{1, 13}=38.817, p<0.001). Asterisks indicate values significantly different between blast- and sham-exposed mice at individual time points (*p<0.05, **p<0.01, unpaired t-tests). For the fear conditioning paradigm (B) results are shown for the training phase, contextual fear memory, which was tested 24 h after training and cued fear memory, which was tested another 24 h later. Pre-tone represents freezing before the first presentation of the tone+/− shock. A repeated measures ANOVA of freezing during the training sessions revealed a significant within-subjects effect of freezing for baseline vs. tone (F_{2.0813, 36.574}=10.425, p<0.001) but no effect of freezing*condition (F_{2.0813, 36.574}=0.203, p=0.883). A test of between-subject effects revealed no significant group differences during the training sessions (F_{1, 13}=0.966, p=0.344). There were no differences between blast-exposed and control groups in the contextual testing (F_{1.742, 19.157}=2.753; p=0.095). In the cued phase testing, neither group showed significant freezing following presentation of the tone (F_{3, 27}=0.790, p=0.510; freezing*condition F_{3, 27}=0.349, p=0.790). However, the blast-exposed exhibited increased freezing compared to the controls (F_{1, 9}=8.758, p=0.016). Error bars in all panels indicate the standard error of the mean (SEM).

FIG. 6. Social preference testing of cohort 1. Blast-exposed (n=7) and control (n=8) mice from cohort 1 were tested in a social preference test. On day 1 (A) the test subjects were first habituated to the apparatus containing two empty metal cups in the side chambers. Time in motion (Move Time) and distance moved (Move Distance) area shown. Tg Sham and Tg Blast mice spent an equal amount of time in motion and moved similar distances. In the pre-test on day 2 (B), subjects were allowed to interact with two non-Tg mice. Time spent in the two chambers (Chamber Time) and total time interacting with the test mice (Interaction Time) are shown. Tg Sham and Tg Blast mice spent an equal amount of time in each chamber (C1 and C2). However, the Tg Blast mice spent more time interacting with the two test mice. Panel (C) shows time interacting with the object and time interacting with unfamiliar test mouse in the test phase on the day 3. Compared to the Tg Sham, the Tg Blast mice spent less time interacting with the object and more time interacting with the test mouse. Error bars in all panels indicate the standard error of the mean (SEM). Asterisks indicate values significantly different between blast- and sham-exposed mice at individual time points (*p<0.05, **p<0.01, unpaired t-tests)

FIG. 7. Elevated zero maze (EZM) testing of cohort 2. APP/PS1 transgenic (Tg) mice were exposed to blast (n=16) or sham (n=16) conditions beginning at 36 weeks of age and received three blast exposures per week for 8 weeks. Behavioral testing was begun at 45 weeks of age (FIG. 1). Time in motion (Move Time), mean speed, total distance traveled (Move Distance), open arm entries, open arm time and the latency to cross into the second open arm (Cross Arm Latency) are displayed. Error bars indicate the standard error of the mean (SEM). Asterisks indicate values significantly different (*p<0.05, unpaired t-tests).

FIG. 8. Novel object recognition (NOR) and Barnes maze testing of cohort 2. Blast-exposed (n=16) and sham-exposed (n=16) APP/PS1 transgenic (Tg) mice from cohort 2 were tested in a novel object recognition (NOR) and Barnes maze. Panel (A) shows time spent exploring the objects (OB1 and OB2) during the NOR training session as well as exploration of the previously presented familiar object (FO) compared to the novel object (NO) when presented 1 h (short-term memory, STM) or 24 h (long-term memory, LTM) later. Panels (B) shows the total time spend exploring the objects during the indicated NOR sessions. Panel (C) shows the latency to enter the escape hole in the Barnes maze. A repeated measures ANOVA revealed a significant within subjects effect by trial (F_{2.731, 76.456}=48.668, p<0.001) but no effect of trial*condition (F_{2.731, 76.456}=1.054, p=0.370) or between subjects effects (F_{1, 28}=0.971, p=0.333). Error bars in all panels indicate the standard error of the mean (SEM). Asterisks indicate values significantly different (*p<0.05, **p<0.01, unpaired t-tests).

FIG. 9. Regression analysis of behavior comparing cohorts 1 and 2. Simple linear regressions were performed comparing cohorts 1 and 2, which were blast exposed beginning at 20 weeks (cohort 1) or 36 weeks (cohort 2) of age. Shown is open arm time (A) or open arm entries (B) in the elevated zero maze (EZM) as well as time spent exploring the novel object in STM (C) or LTM (D) testing of novel object recognition (NOR). p values indicate whether slopes were significantly non-zero.

FIG. 10. Repetitive low-level blast exposure in an Alzheimer's disease transgenic mouse model: Timeline of experiments for cohorts 3 and 4.

FIG. 11. Elevated zero maze (EZM) and light dark (LD) escape testing of cohort 3. APP/PS1 transgenic (Tg) mice were exposed to blast (n=16) or sham (n=16) conditions. Non-transgenic (non-Tg) littermate controls (n=16) were exposed to sham conditions. Mice were subjected to blast or sham conditions beginning at 20 weeks of age and received three blast exposures per week for 8 weeks. The times for behavioral testing are shown in FIG. 8 and Table 1. For the EZM (A) time in motion (Move Time), mean speed, distance moved (Move Distance), open arm entries, time spent in the open arms and the latency to cross into the second open arm (Cross Arm Latency) are shown. In the LD escape task (B), the latency to reach the light center as well as total time spent on the light side and time spent in the light center are shown. Error bars indicate the standard error of the mean (SEM). Overall group differences were compared using a one-way ANOVA. Asterisks indicate significant differences between groups after a significant (p<0.05) one-way ANOVA (*p<0.05, **p<0.01, Fisher's LSD).

FIG. 12. Testing of cohort 3 in novel object recognition (NOR), Barnes maze and fear learning. APP/PS1 transgenic (Tg) mice were exposed to blast (n=16) or sham (n=16) conditions. Non-transgenic (non-Tg) littermate controls (n=16) were exposed to sham conditions. Panel (A) shows time spent exploring the objects (OB1 and OB2) during the NOR training session as well as exploration of the previously presented familiar object (FO) compared to the novel object (NO) when presented 1 h (short-term memory, STM) or 24 h (long-term memory, LTM) later. Panel (B) shows time in motion (Move Time), the latency to find the target quadrant and the latency to enter the escape hole in the Barnes maze. For time in motion, a repeated measures ANOVA revealed a significant within subjects effect by trial (F_{3.697, 162.651}=25.521, p<0.001) but no effect of trial*condition (F_{7.393, 162.651}=0.702, p=0.678) or between subjects effects (F_{2, 44}=1.464, p=0.242). A repeated measures ANOVA of time to find the target quadrant revealed a significant within subjects effect by trial (F_{3.542, 162.651}=48.808, p<0.001) but no effect of trial*condition (F_{7.048, 155.062}=1.971, p=0.062). There were significant between subjects effects (F_{2, 44}=4.314, p=0.019). Post-hoc tests (Fisher's LSD) revealed significant effects for non-Tg Sham vs. Blast Tg (p=0.033) and Sham Tg vs. Blast Tg (p=0.046) but no difference between non-Tg Sham vs. Tg Sham (p=0.981). A repeated measures ANOVA of time to enter the escape hole revealed a significant within-subjects effect by trial (F_{3.286, 141.293}=50.984, p<0.001) but no effect of trial*condition (F_{6.572, 141.293}=2.064, p=0.055). There were significant between-subjects effects (F_{2, 43}=4.312, p=0.020). Post-hoc tests (Fisher's LSD) revealed significant effects for non-Tg Sham vs. Blast Tg (p=0.033) and Tg Sham vs. Tg Blast (p=0.043) but no difference between non-Tg Sham vs. Sham Tg (p=0.986). For the fear conditioning paradigm (C) results are shown for the training phase, contextual fear memory, which was tested 24 h after training and cued fear memory, which was tested another 24 h later. Pre-tone represents freezing before the first presentation of the tone t shock. A repeated measures ANOVA of freezing during the training sessions revealed a significant within-subjects effect of freezing across the training sessions for all groups combined (F_{3.353, 147.533}=33.836, p<0.001) and a significant interaction effect of freezing*condition (F_{6.706, 147.533}=7.570, p<0.001). However, when analyzed alone the Tg Blast mice did not show increased freezing across the trials (F_{2.468, 37.023}=1.036; p=0.378). There were no differences between the groups in the contextual testing (F_{2, 43}=0.473; p=0.626). In the cued phase testing, a repeated measures ANOVA comparing freezing in the pre-tone to first tone across all groups revealed increased freezing (F_{1, 43}=73.436, p<0.001) without interaction effects (F_{2, 43}=0.504; p=0.608). However, there were significant between-subjects effects (F_{2, 43}=6.108, p=0.005). Post-hoc tests revealed significant effects for non-Tg Sham vs. Tg Blast (p=0.002) and non-Tg Sham vs. Tg Sham (p=0.008) but no difference Tg Sham vs. Tg Blast (p=0.594). A repeated measures ANOVA comparing freezing across all groups and all trials revealed increased freezing (F_{4, 172}=20.977, p<0.001) without interaction effects (F_{8, 172}=0.728; p=0.666). However, there were significant between-subjects effects (F_{2, 43}=4.281, p=0.02). Post-hoc tests revealed significant effects for non-Tg Sham vs. Tg Blast (p=0.008) and non-Tg Sham vs. Sham Tg (p=0.032) but no difference between Tg Sham vs. Tg Blast (p=0.551). Error bars in all panels indicate the standard error of the mean (SEM) (*p<0.05, **p<0.01, Fisher's LSD).

FIG. 13. Elevated zero maze (EZM), novel object recognition (NOR) and Barnes maze testing of cohort 4. APP/PSI transgenic (Tg) mice were exposed to blast (n=10) or sham (n=9) conditions. Non-transgenic (non-Tg) littermate controls (n=10) were exposed to sham conditions. For the EZM (A) time in motion (Move Time), mean speed, distance moved (Move Distance), open arm latency, and time spent in the open arms area shown. Panel (B) shows time spent exploring the objects (OB1 and OB2) during the NOR training session as well as exploration of the previously presented familiar object (FO) compared to the novel object (NO) when presented 1 h (short-term memory, STM) or 24 h (long-term memory, LTM) later. Panel (C) shows the total time spent exploring the objects during the indicated NOR sessions. Error bars in all panels indicate the standard error of the mean (SEM). Overall group differences were compared using a one-way ANOVA. Asterisks indicate significant differences between groups after a significant (p<0.05) one-way ANOVA (*p<0.05, **p<0.01, ***p<0.001, ****p<0.0001, Fisher's LSD). Panel (D) shows time to enter the target quadrant in the Barnes maze. A repeated measures ANOVA revealed a significant within-subjects effect by trial (F_{2.306, 57.641}=37.499, p<0.001) but no effect of trial*condition (F_{4.611, 57.641}=2.368, p=0.055). There were significant between-subjects effects (F_{2, 25}=25.178, p<0.001). Post-hoc tests (Fisher's LSD) revealed significant effects for non-Tg Sham vs. Tg Sham (p<0.001), non-Tg Sham vs. Tg Blast (p=0.003) and Tg Blast vs. Tg Sham (p=0.001). A one-way ANOVA of latencies for trial 5 alone revealed significant between-group effects (F_{2, 25}=11.90, p=0.0002). Post-hoc comparisons revealed significant effects for non-Tg vs. Tg Sham (p<0001) and Tg Blast vs. Tg Sham (p=0.0013) but no difference between non-Tg and Tg Blast (p=0.35). Error bars in all panels indicate the standard error of the mean (SEM) (**p<0.01, ****p<0.001, Fisher's LSD).

FIG. 14. Amyloid plaque loads in brains of mice exposed to repetitive low-level blast exposure. Plaque density in the hippocampus was determined in APP/PS1 Tg mice from cohorts 1 and 2 subjected to blast or sham conditions using either thioflavin S staining or immunohistochemical staining with the antibody 6E10. Panel (A) shows representative sections stained with thioflavin S or immunostained with antibody 6E10 from cohort 1. Scale bars=200 μm; insets=10 μm. Panel (B) shows quantitative plaque counts expressed as number per hippocampus. Error bars in all panels indicate the standard error of the mean (SEM). There were no statistically significant differences between the groups.

FIG. 15. Aβ42 levels and Aβ oligomers in the brain of mice exposed to repetitive low-level blast. In panel (A), Aβ42 levels were determined by ELISA in blast- or sham-exposed APP/PS1 Tg mice from cohort 3. In panel (B), Aβ oligomers were determined in the TBS fraction using the same samples studied in panel (A) with antibody A11. A representative dot blot is shown and is quantified in the bar graph. Panel (C) shows Aβ42 in a group of mice from cohort 4 that were euthanized within one week of the last blast exposure. Error bars indicate the standard error of the mean (SEM) (*p<0.05, **p<0.01, ****p<0.0001, unpaired t-tests).

FIG. 16. Correlations between soluble, insoluble and oligomeric Aβ42 with behavioral performance in the EZM. Aβ42 in the TBS (A), Triton X-100 (B) and formic acid (C) fractions as well as oligomeric Aβ42 (D) in APP/PS1 Tg mice from cohort 3 (FIG. 15) were correlated with open arm entries in the EZM (FIG. 11). There were no significant correlations (Table 2).

FIG. 17. Behavioral measures in NOR correlated with soluble, insoluble and oligomeric Aβ42. Aβ42 in the TBS (A), Triton X-100 (B) and formic acid (C) fractions as well as oligomeric Aβ42 (D), determined in APP/PS1 Tg mice from cohort 3 (FIG. 15), were with correlated with data for the STM testing phase of NOR (FIG. 12). There were no significant correlations (Table 2).

FIG. 18. Amyloid beta (Aβ) levels in Triton X-100 (TX) brain fractions (A and B) and PBS fractions (C and D). Levels of Aβ were assessed by ELISA one day (1 d) and 28 days (28 d) after exposure to a single low-intensity blast overpressure of 37 kPa. (A) Exposure to blast was associated with a significant reduction in Aβ 40 levels at the 1 d time point. *p<0.05 vs sham. (B) Aβ 42 levels showed a non-significant ˜14% decrease 1 d after blast compared to sham animals. *p<0.05 vs sham. (C) Aβ 40 levels increased by ˜112% one day post-blast. ****adjusted p<0.0001 vs sham. This peak was significantly reduced 28 d after blast. (D) Blast exposure does not alter Aβ 42 levels in PBS at either time points. Note the differences in the y-axes scales. Values are mean±standard error of the mean (SE).

FIG. 19. Monomeric Aβ levels are not altered after exposure to blast in CSF but are significantly reduced levels in plasma 1 d post-blast. Monomeric Aβ 40 and 42 peptides were determined using an electrochemiluminescent multiplex assay. (A) Aβ 40 and (B) Aβ 42 levels in CSF were examined 1 d and 28 d after blast with no significant changes. (C) Exposure to blast was associated with a ˜35% reduction in plasma Aβ 40 levels at the 1 d time point. ***p<0.001 vs sham, *p<0.05 vs sham. Between sham samples, there was a time effect where 28 d animals showed a significant reduction in Aβ 40 levels compared to 1 d animals. (D) Blast exposure was found to reduce plasma Aβ 42 levels by ˜38% 1 d after blast while levels increased by ˜70% 28 d after blast. **p<0.01 vs sham. A time effect was seen in both sham and blast animals. Plasma Aβ 42 levels significantly decreased with time in sham animals whereas levels significantly increased with time in blast animals. Data are mean concentrations±SE.

FIG. 20 Monomeric Aβ levels are not altered after exposure to blast in CSF but are significantly reduced levels in plasma 1 d post-blast. Monomeric Aβ 40 and 42 peptides were determined using an electrochemiluminescent multiplex assay. (A) Aβ 40 and (B) Aβ 42 levels in CSF were examined 1 d and 28 d after blast with no significant changes. (C) Exposure to blast was associated with a ˜35% reduction in plasma Aβ 40 levels at the 1 d time point. ***p<0.001 vs sham, *p<0.05 vs sham. Between sham samples, there was a time effect where 28 d animals showed a significant reduction in Aβ 40 levels compared to 1 d animals. (D) Blast exposure was found to reduce plasma Aβ 42 levels by ˜38% 1 d after blast while levels increased by ˜70% 28 d after blast. **p<0.01 vs sham. A time effect was seen in both sham and blast animals. Plasma Aβ 42 levels significantly decreased with time in sham animals whereas levels significantly increased with time in blast animals. Data are mean concentrations±SE.

FIG. 21. Exposure to blast does not alter levels of oligomeric Aβ. (A) Levels of oligomeric Aβ in the PBS fraction were assessed by semi-quantitative analysis of dot blot density (mean±SE). AU: arbitrary units. (B) Immunoblots of oligomeric Aβ in cerebral cortex tissue lysate. Blots from representative animals are shown for the time points studied.

FIG. 22. Alterations in the levels of amyloid precursor protein (APP) and APP cleavage products, β-CTF and α-CTF, one 1 d and 28 d after blast exposure in Triton X-100 (TX) and PBS brain fractions. Semi-quantitative analysis of western blot of frontoparietal cortex (mean±SE) showed: (A) a significant ˜21% decrease in APP levels 28 d post-blast in the Triton X-100 fraction. *p<0.05 vs sham.; (B) a 15% reduction APP levels was observed in the PBS fraction 1 d compared to sham animals. *p<0.05; (C) a lack of change in the levels of β-CTF and (D) α-CTF levels in the Triton X-100 fraction. (E) Representative immunoblots of APP, β-CTF, and α-CTF in the Triton X-100 and PBS APP in frontoparietal cortical tissue lysate are shown for each time point studied. β-CTF and α-CTF bands were faint in PBS fractions and are not shown.

FIG. 23. The effects of blast on APP-cleaving secretases. (A) Western blot analysis of levels of BACE-1 in the frontoparietal cortex. Semiquantitative analysis of the blot density shows a trend of a ˜14% decrease 1 d after blast and a ˜11% increase 28 d after blast. (B) BACE1 activity after blast exposure was assessed using a fluorimetric activity assay. BACE1 activity decreased ˜17% blast at the 1 d time point. Kruskal-Wallis followed by Dunn's. **p<0.05 vs sham. Ordinary one-way ANOVA of semiquantitative densitometry analysis of western blot show blast exposure did not alter the levels of (C) PSN1, (D) pro-ADAM10, and (E) active ADAM10 in the frontoparietal cortex at 1 d and 28 d post-blast. All values are means±SE.

FIG. 24. Effect of blast on ADAM17 expression. Semiquantitative analysis of blot density showed that low-intensity blast is associated with a reduction in (A) phosphorylated ADAM17 and (B) total ADAM17 at 1 d and 28 d. The reduction was more significant at the 1 d time point (p<0.0001 for the two forms of the protein). **p<0.01 vs sham. (C) The ratio of phosphorylated-to-total ADAM17 was different between sham and blast-exposed groups (*p<0.05 vs sham), indicating that the decrease in phosphorylated ADAM-17 could be attributed to a reduction in the phosphorylation levels of ADAM-17. (D) ADAM-17 activity was assessed using fluorimetric activity assay, which showed that activity levels of the enzyme in frontoparietal cortical tissue remained unchanged after exposure to blast at both time points.

FIG. 25. Effect of low-intensity blast and ADAM17 alterations on TNF-α. The levels of TNF-α were determined in brain (A) frontopaietal tissue and (B) plasma using an electrochemiluminescent assay. (A) No significant differences in the levels of TNF-α were detected at 1 d and 28 d post-exposure to blast, in spite of a 8-9% reduction in the levels of TNF-α at both study time points. (B) Plasma levels of TNF-α nearly tripled 1 d post-blast (Kruskal-Wallis followed by Dunn's). *p<0.05.

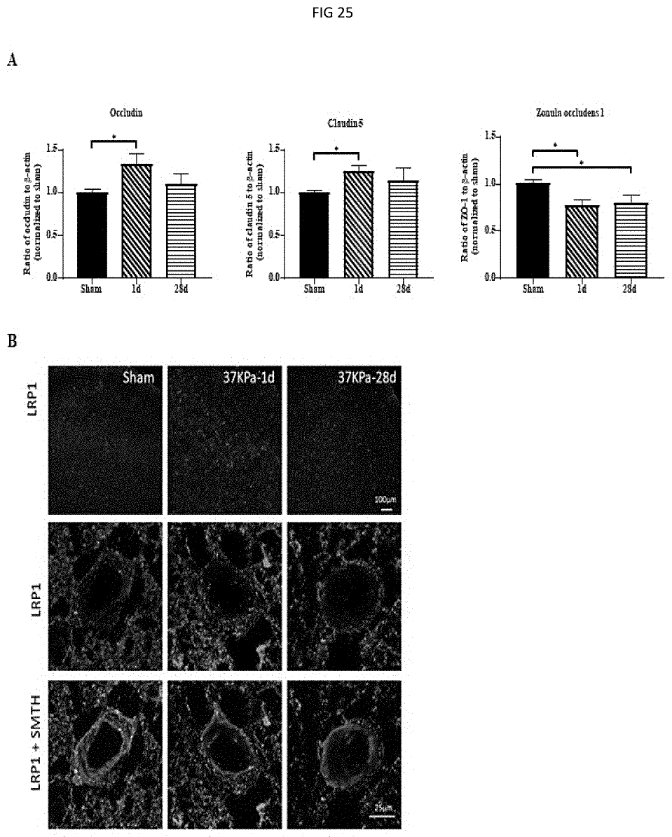

FIG. 26. The effect of blast on the BBB. (A) Western blot analysis of the tight junctional proteins occludin, claudin-5, and zonula occludens-1 (ZO-1) showed dysregulation of some BBB tight junctional proteins. Occludin levels (left) increased 33% 1 d post-blast, claudin 5 increased ˜15% 1 d post-blast, and ZO-1 levels decreased 24% and 20% at 1 d and 28 d post-blast, respectively. *p<0.05. (B) Immunofluorescence of LRP-1 in frontal cortex and in the perivascular space around cortical microvessels was examined using confocal microscopy. An antibody against the vascular smooth muscle protein smoothelin (SMTH) was used to label micovessels. Scale bar=25 μm. (C) Immunofluorescence intensity assessment of LRP-1 immunoreactivity in cortical brain sections demonstrated elevation of LRP-1 signal 1 d after blast exposure. ***p<0.001. (D) Assessment of LRP-1 immunoreactivity in microvessels was also elevated 1 d post blast. **p<0.01.

FIG. 27. Effects of low-intensity blast on AQP4. (A) Cortical expression of AQP4 and GFAP Brain sections from sham- and blast-exposed rats was assessed using immunohistochemistry. Immunofluorescence intensity (arbitrary units; AU) showed that AQP-4 immunofluorescence intensity increased by ˜17% 1 d post-blast and increased ˜19% at 28 d after exposure. ANOVA followed by post-hoc Dunnett's. *p<0.05 vs sham, ***p<0.001. Cortical GFAP immunofluorescence was not altered after blast exposure. (B) Western blotting showed that exposure to blast at the 1 d time point was associated with increase in both the M1 (*p<0.05) and M23 (***p<0.001) isoforms of AQP4. (C) The M1-to-M23 ratio was reduced 1 d after exposure to blast but increased at 28 d after exposure (**p<0.01). Representative western blots of AQP4 isoforms probed with the anti-AQP4 antibody ab125045 are also shown.

FIG. 28. Perivascular expression of AQP4 and GFAP. (A) Brain sections from sham- and blast-exposed rats were probed with antibodies against GFAP, AQP-4, and DAPI to study changes in perivascular expression 1 d and 28 d after blast exposure. Scale bar=25 μm. (B) Analyses of immunofluorescence intensity showed reduction in perivascular expression of AQP4 (left graph) and GFAP (middle graph) 1 d and 28 d post-blast. Co-localization of AQP4 to perivascular GFAP-positive processes (right graph) increased 1 d after blast relative to sham. All comparisons are Kruskal-Wallis followed by Dunn's. ***p<0.001 and ****p<0.0001.

### DETAILED DESCRIPTION OF THE INVENTION

### Definitions

The following definitions are provided to facilitate an understanding of the present invention:

The singular forms “a,” “an,” and “the” include plural referents unless the context clearly dictates otherwise.

As used herein, the terms “subject,” and “patient” refer to any animal, particularly mammals including humans.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., Alzheimer's disease) resulting in a decrease in the probability that the subject will develop the condition or a decrease in the probability progression of the conditions (e.g., in one or more symptoms).

A “effective amount” refers to an amount of treatment effective to prevent, inhibit, or treat a particular disorder or disease and/or the symptoms thereof. For example, “effective amount” may refer to an amount sufficient to inhibit disease progression in a subject.

The term “Neurodegetative Disease”, “Neurodegenerative disorder” refers to a type of disease in which nerve cells in the brain or peripheral nervous system lose function over time and ultimately die. Examples of neurodegenerative disorders include Alzheimer's disease and Parkinson's disease.

The term “Associated Conditions” or “associated symptoms” refer to cognitive deficit or impairment in short term memory, speech, visuospatial skills, and orientation, and difficulty in reasoning or problem-solving, in handling complex tasks or in concentrating, planning or organizing.

The term “Blast overpressure (BOP)”, also known as high energy impulse noise, is a damaging outcome of explosive detonations and firing of weapons. Primary BOP is unique to high-order explosives, results from the impact of the over-pressurization wave with body surfaces.

For purposes of the present invention, the term “subclinical” denotes a disease or a condition, which is not severe enough to present definite or readily observable symptoms. “subclinical” blast is overpressure approximately equal or less than 10 psi and more preferably between 1-10 psi.

For purposes of the present invention, the term “sham” and the term “sham control” refers to the members of a control group that are used to mimic a procedure or treatment without the actual use of the procedure or test substance.

For purposes of the present invention, the term “traumatic brain injury (TBI)” refers to an injury to the brain caused by an external mechanical force.

The present invention relates to a method for treating and/or preventing Alzheimer's disease or other neurodegenerative diseases in a subject, comprising identifying a subject at risk of or in early stage of Alzheimer's disease or other neurodegenerative diseases; and exposing said subject to repetitive low intensity blast overpressure.

Neurodegenerative diseases result from the deterioration of neurons, causing brain dysfunction. The diseases are loosely divided into two groups: conditions affecting memory that are ordinarily related to dementia and conditions causing problems with movements. The most widely known neurodegenerative diseases include Alzheimer (or Alzheimer's) disease and its precursor mild cognitive impairment (MCI), Parkinson's disease (including Parkinson's disease dementia), and multiple sclerosis.

Less well-known neurodegenerative diseases include adrenoleukodystrophy, AIDS dementia complex, Alexander disease, Alper's disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington disease, Kennedy's disease, Krabbe disease, Lewy body dementia, Lyme disease, Machado-Joseph disease, motor neuron disease, Multiple systems atrophy, neuroacanthocytosis, Niemann-Pick disease, Pelizaeus-Merzbacher Disease, Pick's disease, primary lateral sclerosis including its juvenile form, progressive bulbar palsy, progressive supranuclear palsy, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, Tabes dorsalis, Tay-Sachs disease, toxic encephalopathy, transmissible spongiform encephalopathy, Vascular dementia, and X-linked spinal muscular atrophy, as well as idiopathic or cryptogenic diseases as follows: synucleinopathy, progranulinopathy, tauopathy, amyloid disease, prion disease, protein aggregation disease, and movement disorder. A more comprehensive listing may be found at the web site of the National Institute of Neurological Disorders and Stroke (NIDS) of the National Institutes of Health (NIH). It is understood that such diseases often go by more than one name and that a nosology may oversimplify pathologies that occur in combination or that are not archetypical.

Despite the fact that at least some aspect of the pathology of each of the neurodegenerative diseases mentioned above is different from the other diseases, their pathologies ordinarily share other features, so that they may be considered as a group. Furthermore, aspects of their pathologies that they have in common often make it possible to treat them with similar therapeutic methods. Thus, many publications describe features that neurodegenerative diseases have in common.

Among these neurodegenrative disorders, AD is the most prevalent currently affecting 28 million people worldwide. It typically presents with a characteristic amnestic dysfunction associated with other cognitive-, behavioural- and neuropsychiatric changes.

AD is characterized by the abnormal accumulation of intra- and extracellular amyloid deposits, which is closely associated with extensive astrocytosis and microgliosis as well as dystrophic neurones and neuronal loss. These amyloid deposits mainly consist of Aβ-peptides Aβ40 and Aβ42 derived from the Amyloid Precursor Protein (APP), which is expressed on various cell types in the nervous system. Aβ peptides are considered to be directly involved in the pathogenesis and progression of AD.

Besides amyloid deposits, neurofibrillary tangles (NFT) embody the second characteristic neuropathological hallmark of AD. These lesions occur in the hippocampus, amygdale association cortices, and certain subcortical nuclei. NFTs are located in the cytoplasm of neurons and are composed of hyperphosphorylated tau protein. Tau is an axonal, microtubule binding protein that promotes microtubule assembly and stability under normal conditions. Hyperphosphorylation of Tau results in loss of microtubule association and subsequent disassembly of microtubules, which in turn leads to an impairment of axonal transport and subsequent axonal and neuronal degeneration. It is still unclear whether tau hyperphosphorylation and tangle formation are a cause or a consequence of AD.

Besides amyloid and Tau/hyperphosphorylated Tau pathology, neuroinflammation can be considered as the third integral pillar of pathophysiologic changes causing neurodegeneration in AD. The neuroinflammatory phenotype in AD is characterized by robust and widespread activation of microglia and astrocytes in the affected brain regions, resulting in endogenous expression of proinflammatory cytokines, cell adhesion molecules, and chemokines. These changes are thought to result from glial reaction to events related to ongoing toxicity elicited by amyloid and Tau/hyperphosphorylated Tau and their mediators.

Current diagnosis of Alzheimer's disease relies largely on documenting mental decline, at which point, Alzheimer's has already caused severe brain damage. Researchers hope to discover an easy and accurate way to detect Alzheimer's before these devastating symptoms begin. There are several strategies for earlier diagnosis of Alzheimer's disease:

### Biomarkers for Earlier Detection

Experts believe that biomarkers (short for “biological markers”) offer one of the most promising paths. A biomarker is something that can be measured to accurately and reliably indicate the presence of disease. Several potential biomarkers are being studied for their ability to indicate early stages of Alzheimer's disease. Examples being studied include beta-amyloid and tau levels in cerebrospinal fluid (CSF) and brain changes detectable by imaging. Recent research suggests that these indicators may change at different stages of the disease process.

### Brain Imaging/Neuroimaging

Neuroimaging is regularly used today for early detection of Alzheimer's. Structural Imaging provides information about the shape, position or volume of brain tissue. Structural techniques include magnetic resonance imaging (MRI) and computed tomography (CT). Structural imaging such as MRI can reveal tumors, evidence of small or large strokes, damage from severe head trauma, or a buildup of fluid in the brain, as well as detect underlying conditions that may preclude an individual from certain treatments. Brains of people with Alzheimer's disease have been shown to shrink significantly as the disease progresses, structural imaging research also has shown that shrinkage in specific brain regions such as the hippocampus may be an early sign of Alzheimer's.

Functional imaging reveals how well cells in various brain regions are working by showing how actively the cells use sugar or oxygen. Functional techniques include positron emission tomography (PET) and functional MRI (fMRI). Functional imaging research suggests that those with Alzheimer's typically have reduced brain cell activity in certain regions. For example, studies with fluorodeoxyglucose (FDG)-positron emission tomography (PET) indicate that Alzheimer's is often associated with reduced use of glucose (sugar) in brain areas important in memory, learning and problem-solving. According to Medicare recommendations, an FDG-PET scan is considered a reasonable test for people with a recent diagnosis of dementia and documented cognitive decline of at least six months who meet diagnostic criteria for both Alzheimer's and frontotemporal dementia.

Molecular imaging uses highly targeted radiotracers to detect cellular or chemical changes linked to specific diseases. Molecular imaging technologies include PET and fMRI. Molecular imaging, which also uses PET scans, is among the most active areas of research aimed at finding new approaches to diagnose Alzheimer's in its earliest stages. Molecular strategies may detect biological clues indicating Alzheimer's is under way before the disease changes the brain's structure or function, or takes an irreversible toll on memory, thinking and reasoning. Molecular imaging also may offer a new strategy to monitor disease progression and assess the effectiveness of next-generation, disease-modifying treatments. Several molecular imaging compounds are being studied, and four have been approved for clinical use. Florbetaben (NEURACEQ®), Florbetapir (AMYVID®) and Flutemetamol (VIZAMYL®) have been approved for detection of beta-amyloid in the brain. Flortaucipir F18 (TAUVID®) has been approved for detection of tau in the brain.

Today, a diagnosis of Alzheimer's is based on the evaluation of several things, including the presence of amyloid plaques. Your doctor may perform tests to evaluate your memory, order laboratory tests or perform a molecular imaging test (e.g., PET scan) to confirm an Alzheimer's diagnosis or rule out other diseases that may cause similar symptoms.

### Cerebrospinal Fluid (CSF) Tests

CSF is a clear fluid that bathes and cushions the brain and spinal cord. Adults have about 1 pint of CSF, which physicians can sample through a minimally invasive procedure called a lumbar puncture, or spinal tap. Research suggests that Alzheimer's disease in early stages may cause changes in CSF levels of multiple markers such as tau and beta-amyloid, two markers that form abnormal brain deposits strongly linked to Alzheimer's. Another potential marker is neurofilament light (NfL), an increased level of which has been found in neurodegenerative diseases such as Alzheimer's. CSF tests are currently used by dementia specialists to aid in the diagnosis of Alzheimer's, and research continues to develop and standardize new markers that will aid in diagnosis and detection of other dementias. One CSF Amyloid Ratio test, LUMIPULSE®, received FDA approval and is a new diagnostic tool that clinicians can use to detect amyloid in CSF, which can be predictive of amyloid changes in the brain.

### Blood Tests

Researchers are investigating whether consistent and measurable changes in blood levels of specific markers may be reliably associated with Alzheimer's related changes. These markers may include tau, beta-amyloid or other biomarkers that could be measured before and after symptoms appear. There are a few blood tests currently on the market that can be ordered by health care providers to aid in the diagnosis of memory complaints. These tests do not yet have FDA approval. These blood tests cannot be used as a stand-alone test to diagnose Alzheimer's disease or any other dementia; they will be used as part of a diagnostic workup with other exams.

### Genetic Risk Profiling

Scientists have identified three genes with rare variations that cause Alzheimer's (Dominantly Inherited Alzheimer's Disease) and several genes that increase risk but don't guarantee that a person will develop the disease. Investigators worldwide are working to find additional risk genes as well those that may decrease an individual's risk. Genetic testing for APOE-e4, the strongest risk gene in some populations, is included in some clinical trials to identify participants at high risk for Alzheimer's disease or risk side effects that may be associated with approved treatments.

One or more of these above mentioned techniques may be used alone or in combination to identify subject at risk or in early stage if Alzheimer's Disease and/or other neurodegerative disease.

Application of shock waves in medicine is not new. Extracorporeal-generated shock waves were first introduced disintegrate kidney stones. This treatment method substantially changed the treatment of urolithiasis. Shock waves have become the treatment of choice for kidney and ureteral stones. Urology, however, is not the only medical field for the potential use of shock waves. Shock waves subsequently have been used in orthopaedics and traumatology to treat various insertional tendinopathies (enthesiopathies) and delayed unions and nonunions of fracture. Shock wave application also has been used in the treatment of tendinopathies in veterinary conditions (race horses). In present invention, repetitive low intensity blast overpressure (pulse shock waves) are administered to a subject at risk of or in early stage if Alzheimer's Disease and/or other neurodegerative disease for a period of time. The intensity of the blast overpssure is maintained at a subclinical level, at approximately equal or less than 10 psi). Preferably at approximately 1-10 psi. The subclinical blast overpressure is administered intermittently (a couple of time per week) for a prolong period (e.g. weeks).

In one embodiment, the inventive method improve behavioral deficits. For example, the inventive method may improve behavioral deficits include but not limited to anxiety, impaired cognition, social interactions, loss of spatial, impairment in short term memory, speech impediment, visuospatial skills impairment, orientation impairment, and difficulty in reasoning or problem-solving, difficulty in handling complex tasks, difficulty in concentrating, planning and organizing or a combination thereof.

In another embodiment, the inventive method reduces abnormal accumulation of brain proteins in a subject at risk or in early stage of Alzheimer's disease or other neurodegenerative diseases. Such brain proteins include but not limited to α-synuclein, tau, amyloid precursor protein (APP), amyloid β protein (Aβ) or a combination thereof. The amyloid β protein (Aβ) may be Aβ 42 or Aβ 40. The reduction in abnormal accumulation of brain proteins is achieved via reduced processing of amyloid β protein (Aβ) and/or altering enzymatic, transvascular, and perivascular clearance of Aβ.

In yet another embodiment, the inventive method improves of brain inflammation, decreases APP-cleaving secretases, increasing trans-endothelial clearance via LRP1, and or improves paravascular glymphatic AQP4-mediated clearance. The inventive method may be used in combination with one or more therapy or therapeutic agents for Alzheimer's disease or other neurodegenerative diseases. The therapeutic agents may be administered to the subject at an effective amount to prevent β-amyloid deposition, reduce β-amyloid production, improve β-amyloid clearance, improve brain inflammation, or inhibit of BACE1. Some examples of such agents are disclosed in US Patent Publication US20170049810A1, U.S. Pat. No. 8,097,259B2, US20100144790A1, U.S. Ser. No. 10/118,4982. Two exemplary therapies for Alzheimer's disease using magnetic or electric stimulation are disclosed in U.S. Patent Publication U.S. Pat. No. 9,233,258B2 and US20220288385A1.

### Example 1: Repetitive Low-Level Blast Exposure in an Alzheimer's Disease Transgenic Mouse Model

### Materials and Methods

### Animals

APP/PS1 transgenic (Tg) mice [Tg(APPswe, PSEN1dE9)85Dbo; Stock No. 34829-JAX] were obtained from the Jackson Laboratory on a C57BL/6;C3H genetic background. All studies involving animals were reviewed and approved by the Institutional Animal Care and Use Committees of the Walter Reed Army Institute of Research (WRAIR)/Naval Medical Research Center and the James J. Peters VA Medical Center. Studies were conducted in compliance with the Public Health Service policy on the humane care and use of laboratory animals, the NIH Guide for the Care and Use of Laboratory Animals, and all applicable Federal regulations governing the protection of animals in research.

### Blast Overpressure Exposure

Mice were exposed to overpressure injury using a shock tube, which simulates the effects of air blast exposure under experimental conditions. The shock tube has a 12-inch circular diameter and is a 19.5 ft-long steel tube divided into a 2.5 ft compression chamber that is separated from a 17 ft expansion chamber. The compression and expansion chambers are separated by polyethylene MYLAR™ sheets (Du Pont, Wilmington, Del., USA) that control the peak pressure generated. The peak pressure at the end of the expansion chamber was determined by piezoresistive gauges specifically designed for pressure-time (impulse) measurements (Model 102M152, PCB, Piezotronics, Depew, N.Y., USA).

Individual mice were anesthetized using an isoflurane gas anesthesia system consisting of a vaporizer, gas lines and valves and an activated charcoal scavenging system adapted for use with rodents. Mice were placed into a polycarbonate induction chamber, which was closed and immediately flushed with 5% isoflurane in air mixture for two minutes. To eliminate rotational/accelerational injury during exposure to blast, mice were placed side-by-side along the center (horizontal) axis of the circular (10-inch diameter) rodent constraint device. The rodents were held in place between two layers of fabric that were secured in place between the two rings of the device by four clasps, one at each corner. The constraint device was then secured in place with the animals on their stomachs and facing into the shock tube 10 inches from the end of the shock tube. Each subject to receive blast exposure was exposed to one 34.5-kPa exposure a day for three days in a row, followed by four days of no exposure, for a total of eight weeks. Sham animals received isoflurane and were placed in the device and the shock tube for the same amount of time as the blast-exposed animals but were not exposed to blast. Within 10 days after the last blast or sham exposure animals were transported in a climate-controlled van to the James J. Peters VA Medical Center (Bronx, N.Y., USA). Animals were shipped in the morning from NMRC arrived in the afternoon of the same day at the James J. Peters VA Medical Center, where all other procedures were performed.

### Animal Housing

Animals were housed at a constant 70-72° F. temperature with rooms on a 12:12 hour light cycle with lights on at 7 AM. All subjects were individually housed in standard clear plastic cages equipped with Bed-O'Cobs laboratory animal bedding (The Andersons, Maumee, Ohio, USA) and EnviroDri nesting paper (Sheppard Specialty Papers, Milford, N.J., USA). Access to food and water was ad libitum. Subjects were housed on racks in random order to prevent rack position effects. Cages were coded to allow maintenance of blinding to groups during behavioral testing.

### Behavioral Testing

### Elevated Zero Maze

The apparatus consisted of a circular black Plexiglas runway 61 cm in diameter and raised 61 cm off the floor (San Diego Instruments, San Diego, Calif., USA). The textured runway itself was 5.0 cm across and divided equally into alternating quadrants of open runway enclosed only by a 0.80-cm lip and closed runway with smooth 15.5-cm walls. All subjects received a 5-min trial beginning in a closed arc of the runway. During each trial, subjects were allowed to move freely around the runway, with all movement tracked automatically by a video camera placed on the ceiling directly above the maze. Data were analyzed by ANYMAZE (San Diego Instruments, San Diego Calif., USA) yielding measures of total movement time and distance for the entire maze, as well as time spent and distance traveled in each of the individual quadrants. From the quadrant data, measures of total open and closed arc times, latency to enter an open arc, total open arm entries and latency to completely cross an open arc between two closed arcs were calculated. Subject position was determined by centroid location.

### Light/Dark Emergence

A light/dark emergence task was run in Versamax activity cages with opaque black Plexiglas boxes enclosing the left half of the interiors so that only the right sides were illuminated. Animals began in the dark side and were allowed to freely explore for 10 min with access to the left (light) side through an open doorway located in the center of the monitor. Subject side preference and emergence latencies were tracked by centroid location with all movement automatically tracked and quantified. Light-side emergence latency, time to reach the center of the lighted side (light-side center latency) and percent total light-side duration were calculated from beam breaks. All equipment was wiped clean between tests.

### Novel Object Recognition (NOR)

Mice were habituated to the circle arena (30 cm length×30 cm width×40 cm height) for 10 min, 24 h before training. On the training day, two identical objects were placed on opposite ends of the empty arena, and the mouse was allowed to freely explore the objects for 7 min. After 1 h, during which the mouse was held in its home cage, one of the two familiar objects (FOs) was replaced with a novel object (NO) and the mouse was allowed to freely explore the familiar and NO for 5 min to assess short-term memory (STM). After 24 h, during which the mouse was held in its home cage, one of the two FOs was replaced with a NO different from the one used during the STM test. The mouse was allowed to freely explore the familiar and NO for 5 min to assess long-term memory (LTM). Raw exploration times for each object were expressed in seconds. Object exploration was defined as sniffing or touching the object with the vibrissae or when the animal's head was oriented toward the object with the nose placed at a distance of <2 cm from the object. All sessions were recorded by video camera (Sentech, Carrollton, Tex.) and analyzed with ANYMAZE software (San Diego Instruments). In addition, offline analysis by an investigator blind to the treatment status of the animals was performed. Objects to be discriminated were of different size, shape and color, and were made of Lego plastic material. All objects were wiped with 70% ethanol between trials. A discrimination index (DI) was calculated with the formula: time exploring the novel object minus time exploring the familiar object/total exploration time×100.

### Novel Object Localization

Novel object localization was assessed using methods previously described. Twenty four h before training, mice were habituated for 20 min to the same empty arena used for the NOR task. The arena was situated in a well-lit room allowing the rats to see distal visual cues. On the training day, two identical objects were placed in specific locations and the mouse was allowed to freely explore the objects for 7 min. The test trial was performed after a 1-h delay during which one object was moved to a different location in the arena and the mouse was allowed to explore for 5 min. Time spent investigating the objects in their original or novel locations was recorded. During sessions the arena and objects were cleaned before and between trials with 70% ethanol.

### Barnes Maze

The Barnes maze test was performed using a standard apparatus. The testing was conducted in two phases: training (day 1 to 4) and testing (day 5). Before starting each experiment, mice were acclimated to the testing room for 1 h. Mice were transported from their cage to the center of the platform with a closed starting chamber where they remained for 10 s before exploring the maze. Mice failing to enter the escape box within 4 min on trials 1-4 were guided to the escape box by the experimenter and the latency was recorded as 240 s. Trial 5 was treated as a test trial and mice were given up to 180 s to enter the escape box. The platform and the escape box were wiped with 70% ethanol after each trial. Trials were recorded by video camera and analyzed with ANYMAZE software.

### Contextual and Cued Fear Conditioning

Sound-attenuated isolation cubicles (Coulbourn Instruments, Holliston, Mass., USA) were utilized. Each cubicle was equipped with a grid floor for delivery of the unconditioned stimulus (US) and overhead cameras. All aspects of the test were controlled and monitored by the Freeze Frame conditioning and video tracking system (Actimetrics, Coulbourn Instruments). During training the chambers were scented with almond extract, lined with white paper towels, had background noise generated by a small fan and were cleaned before and between trials with 70% ethanol. Each subject was placed inside the conditioning chamber for 2 min before the onset of a conditioned stimulus (CS; an 80 dB, 2 kHz tone), which lasted for 20 s with a co-terminating 2-s footshock (0.7 mA; unconditioned stimulus [US]). A total of three tone/shock pairings were administered with the first/second and second/third separated by 1 min. Each mouse remained in the chamber for an additional 40 s following the third CS-US pairing before being returned to its home cage. Freezing was defined as a lack of movement (except for respiration) in each 10-s interval. Minutes 0-2 during the training session were used to measure baseline freezing. Contextual fear memory testing was performed 24 h after the training session by measuring freezing behavior during a 4-min test in the conditioning chamber under conditions identical to those of the training session with the exception that no footshock or tone (CS or US) was presented. Animals were returned to their home cage for another 24 h at which time cued conditioning was tested. To create a new context with different properties, the chambers were free of background noise (fan turned off), lined with blue paper towels, scented with lemon extract and cleaned before and during all trials with isopropanol. Each subject was placed in this novel context for 2 min and baseline freezing was measured, followed by exposure to the CS (20-s tone) at 120 and 290 s.

### Social Preference Test

A three-chamber social preference test was used to assess preference for social vs. non-social stimuli. The test was modeled after other published protocols. The apparatus consisted of a grey opaque polycarbonate rectangle (64×41×25 cm) that was divided into three chambers using removable partitions. Each divider (41×21 cm) had a sliding door of≈5×5 cm to allow free movement of the animal between chambers. The central chamber served as the starting area while the lateral chambers were used to hold a stimulus. The mouse stimulus was placed in a metallic cage/jail of height 15 cm having a diameter of 7 cm which allowed interactions between the test subject and mouse stimulus but limited aggressive interactions. The protocol comprised three phases that were completed over 3 days. On day 1 the test subject was first habituated to the apparatus containing two empty metal cups in the side chambers. The test subject was allowed to freely explore the 3 chambers for 10 min and basal activity was recorded. In the pre-test phase on day 2, the subject was allowed to interact with two non-Tg mice of the same age as the test subject (one in each metal cup) for 5 min. During the test phase on the day 3, the test subject was given the choice of interacting with a new mouse (unfamiliar non-Tg) contained in one cup or a novel non-social stimulus (an object) contained in the other cup for 5 min. Movement of the test subject was tracked by ANYMAZE software recording the time in motion, distance moved, entries and exits from the chambers as well as time interacting/sniffing the object or the jailed mouse.

### Aβ Enzyme-Linked Immunosorbent Assay (ELISA)

Animals were euthanized by CO₂ narcosis and the brains were quickly removed, frozen and stored at −80° C. until use. TBS, Triton X-100 and formic acid fractions from one hemisphere, were prepared using a protocol adapted from that described in Kawarabayashi et al. and described in more detail by Steele et al. The tissues were homogenized with a hand held homogenizer in 50 mM Tris-HCl buffer, pH 7.4, 150 mM NaCl (TBS) with a protease/phosphatase inhibitors cocktail (ThermoFisher. Waltham, Mass.) (200 mg tissue/ml) and 0.25 ml were centrifuged at 100,000 g for 1 h at 4° C. The supernatant was saved (TBS fraction) and the pellet homogenized with 1% Triton X-100 in TBS supplemented with protease/phosphatase inhibitor cocktail (ThermoFisher). The supernatant was saved (Triton fraction) and the pellet extracted with ice-cold 70% formic acid and centrifuged as above. The supernatant was saved (formic acid fraction). Aβ42 levels in every fraction were determined by ELISA using a commercially available kit that detects human Aβ42 (Wako, Richmond, Va.). Data are expressed as pg/mg fresh tissue.

### Oligomeric Aβ42 Dot Blot Analysis

Oligomeric Aβ42 was determined by dot blot analysis. Protein concentration was determined with the BCA reagent (ThermoFisher). An aliquot containing 2.5 mg protein was spotted onto a nitrocellulose membrane and the membrane was air-dried, washed in TBS and blocked for 1 h in TBS/5% non-fat dry milk. The membrane was then incubated for 1 h with anti-oligomer antibody A11 (1:1500, #AHB0052, ThermoFisher), washed in TBS and incubated for 1 h with horseradish peroxidase-conjugated anti-rabbit antibody (1:10,000, #NA-934, Cytiva Lifesciences, Marlborough, Mass.) diluted in blocking solution. The immunoreactive signal was visualized with ECL Prime reagent (Cytiva Lifesciences), imaged with an Amersham Image Quant 1200 imaging station and quantitated by ImageQuantTL software (Cytiva Lifesciences). Data were normalized to sham samples.

### Immunohlstochemistry

Mice were perfused with 4% paraformaldehyde in PBS, and the brains dissected and post-fixed overnight in 4% paraformaldehyde. The brains were sectioned into 40 mm-thick coronal sections with a Vibratome (Leica, Wetzlar, Germany). For stereologic analyses, sections that contained the entire hippocampus were selected every 300 mm (interaural 0.72-1.44 mm) from 6 control and 6 blast-exposed APP/PS1 Tg animals. Amyloid plaques were identified by immunohistochemical staining with the mouse monoclonal antibody 6E10 (1:1,000, LSBio #LS-C821449, Seattle, Wash.), which recognizes an epitope in the N-terminal region of both Aβ40 and Aβ42. Sections were blocked with TBS/0.3% Triton X-100, 5% normal goat serum for 1 h and stained overnight with primary antibodies diluted in blocking solution. The sections were washed in PBS and incubated for 2 h with the appropriate Alexa-fluor-conjugated secondary antibody (1:300, ThermoFisher) in blocking solution. After washing with PBS the sections were mounted in FluoroGel mounting medium (EMS Science, Hatfield, Pa.). Total plaque number in the hippocampal region in each section was determined using a Zeiss Axioplan 2 microscope at 40× magnification under UV illumination.

### Thioflavin S Staining

Sections were incubated in 1% aqueous Thioflavin S (Sigma-Aldrich, St. Louis, Mo.) for 8 min at room temperature in the dark. Sections were washed twice for 3 min in 80% ethanol, 3 min with 95% ethanol, rinsed three times with distilled water and mounted with Fluorogel. Sections were sampled as above and the total number of Thioflavin S positive plaques in the hippocampal areas was determined.

### Statistical Analysis

Values are expressed as means±the standard error of the mean (SEM). The groups and group sizes are indicated in Table 1. Data sets were tested for normality using the D'Agostino-Pearson normality test. Comparisons were performed using repeated-measures ANOVA, one-way ANOVA or unpaired t-tests. When repeated-measures ANOVA was used sphericity was assessed using Mauchly's test. If the assumption of sphericity was violated (p<0.05), significance was determined using the Greenhouse-Geisser correction. Between-group comparisons after a significant one-way ANOVA were compared using Fisher's LSD. For some comparisons, simple linear regressions were performed or Pearson's product-moment correlation coefficient, Kendall's tau-b, and Spearman's rho were calculated. Statistical tests were performed using the programs GraphPad Prism 8.0 (GraphPad Software, San Diego, Calif.) or SPSS v26 (IBM, Armonk, N.Y.).

TABLE 1
Summary of behavioral testing in blast-exposed mice

| Cohorts | Cohort 1 | Cohort 2 | Cohort 3 | Cohort 4 |
|---|---|---|---|---|
| Age at time | 20 weeks | 36 weeks | 20 weeks | 20 weeks |
| blast exposure | | | | |
| was initiated | | | | |
| Groups and | Tg Blast (7) | Tg Blast (16) | Tg Blast (16) | Tg Blast (10) |
| group sizes | Tg Sham (8) | Tg Sham (16) | Tg Sham (16) | Tg Sham (9) |
| (n) | | | non-Tg Sham (16) | non-Tg Sham (10) |
| Locomotor | Tg Blast exhibited | No differences | Not tested | Not tested |
| activity | increased center | between Tg Blast | | |
| | time compared to Tg | and Tg Sham | | |
| | Sham | | | |
| Elevated zero | Tg Blast exhibited | Tg Blast exhibited | Blast rescued anxiety | Blast rescued |
| maze | less anxiety than Tg | less anxiety than Tg | phenotype found in | anxiety phenotype |
| | Sham | Sham | Tg sham mice | found in Tg sham |
| | | | | mice |
| Light dark | Tg Blast exhibited | No differences | Blast rescued anxiety | Not tested |
| escape | less anxiety than Tg | between Tg Blast | phenotype found in | |
| | Sham | and Tg Sham | Tg sham mice | |
| Novel object | Deficits in NOR in | Deficits in NOR in | Deficits in NOR in | Deficits in NOR in |
| recognition | Tg Sham mice were | Tg Sham mice were | Tg Sham mice were | Tg Sham mice were |
| | rescued in Tg Blast | rescued in Tg Blast | rescued in Tg Blast | rescued in Tg Blast |
| | mice. | mice. | mice. | mice. |
| Novel Object | Deficits in NOL in | Not tested | Not tested | Not tested |
| Localization | Tg Sham mice were | | | |
| | rescued in Tg Blast | | | |
| | mice | | | |
| Barnes Maze | Tg Blast mice showed | No differences in | Tg Blast exhibited | Tg Blast exhibited |
| | improved learning | performance of Tg | better learning | better learning |
| | curves compared to | Blast vs. Tg Sham | curves than either | curves than either |
| | Tg Sham mice | | non-Tg Sham or Tg | non-Tg Sham or Tg |
| | | | Sham mice. | Sham mice. |
| Fear | Tg Blast froze more | Neither Tg Blast nor | Tg Blast mice failed | No tested |
| conditioning | than Tg Sham in the | Tg Sham formed an | to form an association | |
| | cued phase | association between | between the tone | |
| | | the tone and the | and the shock during | |
| | | shock during the | the training session | |
| | | training session | | |
| Social | Blast improved social | No differences in | Not tested | Not tested |
| interaction | interactions in Tg | social interactions | | |
| | Blast vs. Tg Sham | of Tg Blast and Tg | | |
| | mice | Sham mice | | |

Results highlighted in BOLD reflect tests where Tg Blast mice performed better than Tg Sham.

### Results

### Experimental Design for Blast Exposure of APP/PSI Tg Mice.

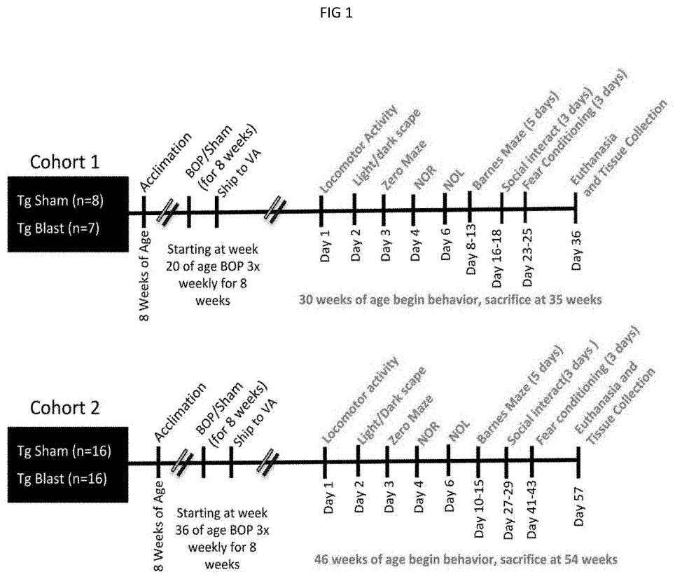

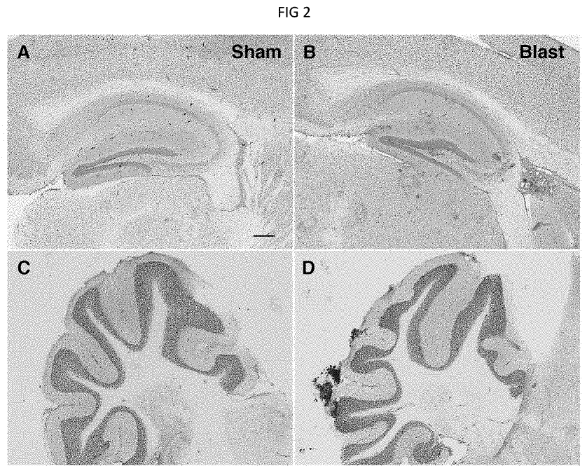

To determine the effects of an extended blast exposure protocol on APP/PS1 Tg mice, APP/PS1 Tg mice exposed to sham or blast conditions were compared. FIG. 1 shows the experimental design and timeline of the first two experiments. The groups and group sizes are indicated in Table 1. Blast-exposed mice received one 34.5-kPa exposure a day for three days in a row, followed by four days of no exposure, for a total of eight consecutive weeks. Exposures began at 20 weeks of age (cohort 1), an age before APP/PS1 Tg develop substantial plaque loads, or 36 weeks (cohort 2), when significant plaque burdens are present. Sham-exposed control mice were treated identically to those blast-exposed, including receiving anesthesia and being placed in the blast tube but did not receive a blast exposure. The timing of the studies for cohorts 1 and 2 are shown in FIG. 1. Histopathologic inspection using Nissl staining did not reveal any consistent anatomical abnormalities in blast-exposed animals compared to shams (FIG. 2). Behavioral test results for cohorts 1 and 2 are summarized in Table 1.

Repetitive low-level blast exposure reduces anxiety and improves cognition as well as social interactions in APP/PS1 Tg mice when begun at 20 weeks of age.

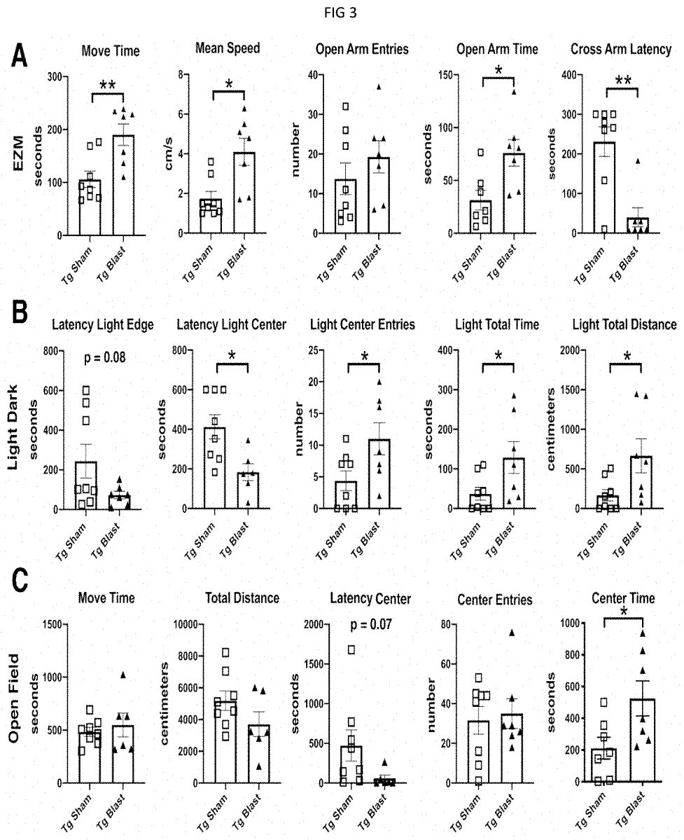

FIG. 3 shows testing of sham and blast-exposed APP/PS1 Tg mice from cohort 1 in tests that measure anxiety. In an elevated zero maze (EZM, FIG. 3A), blast-exposed APP/PS1 Tg mice from cohort 1 spent more time in motion and moved faster, as well as spent more time in the open arms and exhibited a shorter latency to cross into the second open arm (cross arm latency). In the light/dark escape task (L/D, FIG. 36) blast-exposed APP/PS1 Tg mice exhibited a shorter latency to reach the light center and made more light center entries as well as spent more time and traveled a greater distance on the light side. Compared to sham-exposed mice, in an open field test (FIG. 3C), blast-exposed APP/PS1 Tg mice spent more time in the center of the open field. All these results suggest that blast-exposed APP/PS1 Tg mice exhibit less anxiety compared to sham-exposed APP/PS1 Tg mice.

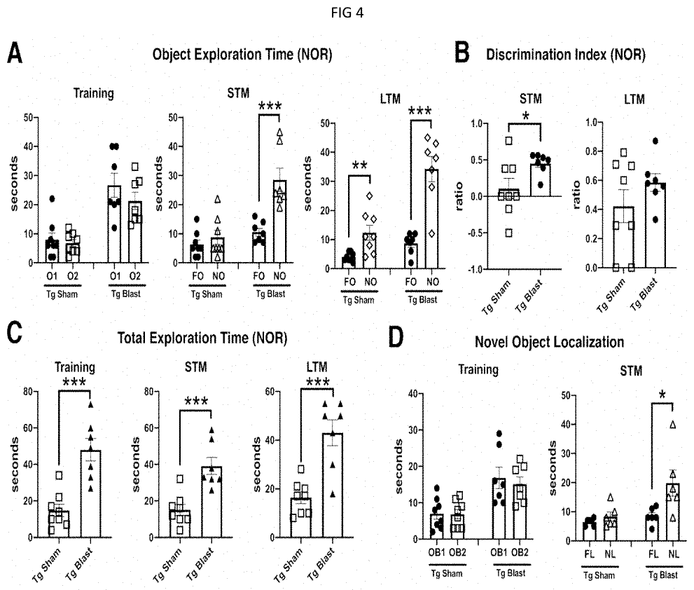

FIG. 4 shows testing of mice from cohort 1 in novel object recognition (NOR) and novel object localization (NOL) tasks. In the NOR training session, sham and blast-exposed APP/PS1 Tg mice spent comparable time exploring the two objects that had not been previously encountered (FIG. 4A) although blast-exposed APP/PS1 Tg mice spent more total time exploring the objects (FIG. 4C). In STM testing (FIG. 3A), blast-exposed APP/PS1 Tg spent more time exploring the novel object (NO) compared to the familiar object (FO) indicating intact recognition memory, unlike the sham-exposed APP/PS1 Tg who explored the NO no more than the FO, indicating a failure of recognition memory. Blast-exposed APP/PS1 Tg also showed an increased preference for the NO vs. FO when a discrimination index was calculated for the STM testing (FIG. 4B), which relates the relative tendency to explore the NO vs. FO. In LTM testing, both blast-exposed and sham-exposed APP/PS1 Tg mice preferentially explored the NO vs. FO, suggesting that with repeated presentation of the FO, recognition memory improved in the sham-exposed mice. However, as in the other testing sessions, blast-exposed APP/PS1 Tg mice spent more total time exploring the objects in the LTM testing (FIG. 4C).

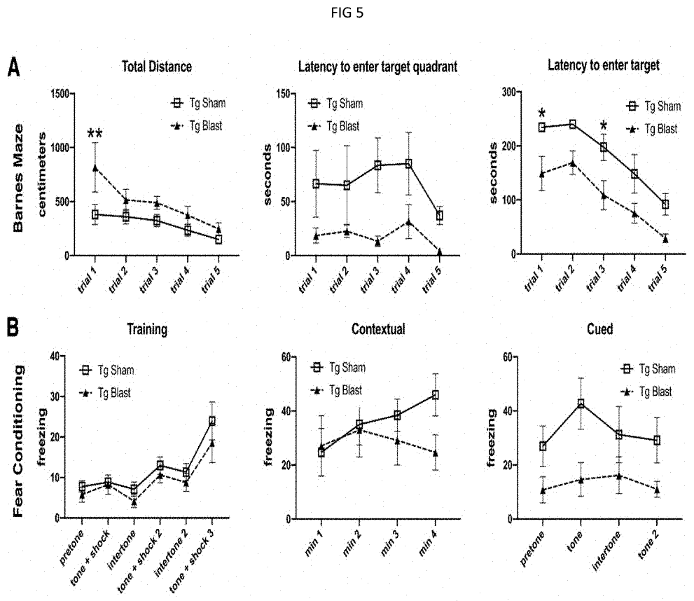

In a NOL test, when tested 24 h after the training session, blast-exposed APP/PS1 Tg mice explored the object moved to the novel location more, unlike sham-exposed APP/PS1 Tg which explored both objects equally indicating that APP/PS1 Tg mice exposed to blast recognized the change in location of the object while sham exposed APP/PS1 Tg mice did not. Thus blast-exposed APP/PS1 Tg mice showed intact recognition memory in both NOR and NOL tasks compared to sham-exposed controls which were impaired in both tasks. Testing of cohort 1 in a Barnes maze showed that blast-exposed APP/PS1 Tg mice exhibited faster learning curves and shorter latencies to enter the escape hole than sham-exposed APP/PS1 Tg mice (FIG. 5A). Thus, blast-exposed APP/PS1 Tg mice exhibited better cognition than sham exposed APP/PS1 Tg mice across multiple tests.

FIG. 5B shows testing of cohort 1 in a fear-learning paradigm. In the training phase, both blast- and sham-exposed APP/PS1 Tg mice exhibited similar learning curves showing increased freezing after repetitive presentation of the tone/shock pairing. There were no differences between blast-exposed and sham groups in the contextual testing. In the cued phase testing, neither group showed significant freezing following presentation of the tone. However, the blast-exposed APP/PS1 Tg mice exhibited overall increased freezing compared to the controls, indicating that while impaired cued fear learning was present in both groups, in this task, blast exposure altered the general freezing tendency of APP/PS1 Tg mice compared to sham-exposed APP/PS1 Tg mice.

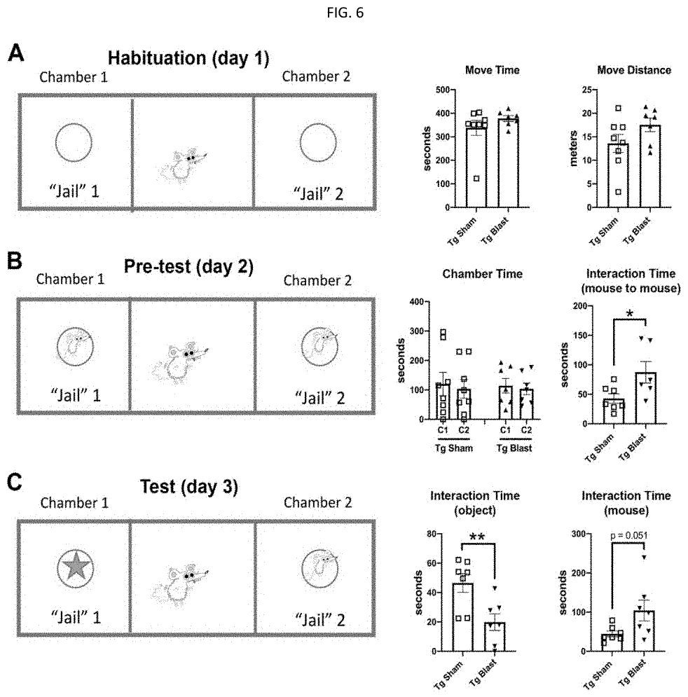

FIG. 6 shows social interaction testing of cohort 1. During the habituation phase on day 1 sham- and blast-exposed Tg mice spent an equal amount of time in motion and moved similar distances exploring the empty chambers (FIG. 6A). On day 2, when presented with two unfamiliar test mice in different chambers, sham- and blast-exposed mice spent an equal amount of time in each chamber (FIG. 6B). However, the blast-exposed mice spent more total time interacting with the test mice (FIG. 6B). In the test phase on day 3 (FIG. 6C) when given the choice of exploring an object or unfamiliar test mouse, the Tg Blast mice spent less time interacting with the object and more time interacting with the mouse compared to the Tg Sham. Thus, repetitive low-level blast exposure improves social interactions in APP/PS1 Tg mice when initiated at 20 weeks of age.

Repetitive low-level blast exposure is less effective at Improving behavioral deficits in APP/PS1 Tg mice when begun at 36 weeks of age.

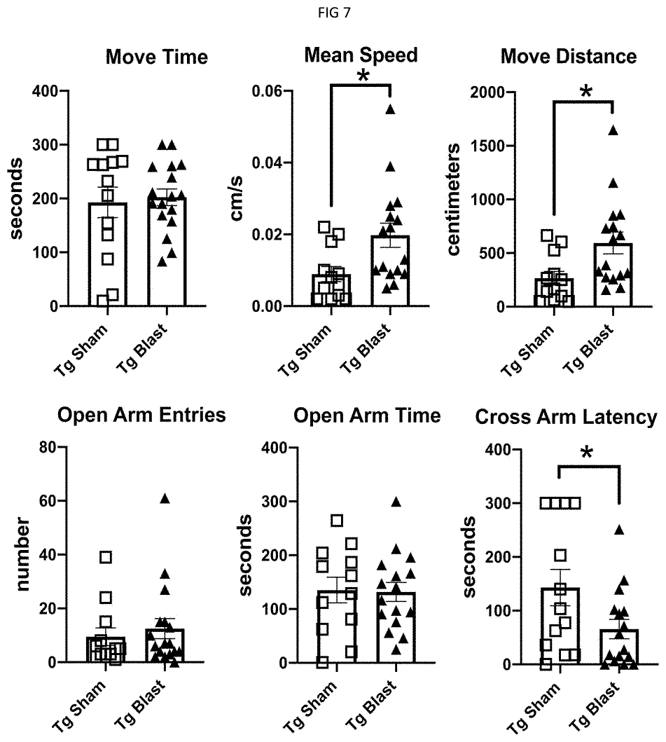

Cohort 2 began blast exposure at 36 weeks of age, a time at which significant plaque burdens are established in APP/PS1 Tg mice³⁹. When studied between 53 and 62 weeks of age (FIG. 1), there were no differences in locomotor activity between sham- and blast-exposed APP/PS1 Tg mice in an open field. In a L/D escape task while there was a trend for blast-exposed APP/PS1 Tg mice to make more entries into the light center and spend more time on the light side, these trends did not reach statistical significance (p=0.06, unpaired t-tests in both parameters). In the EZM, as with cohort 1 (FIG. 3), blast-exposed APP/PS1 Tg mice of cohort 2 moved more and exhibited shorter cross arm latencies although they did not differ from sham-exposed in open arm time (FIG. 7).

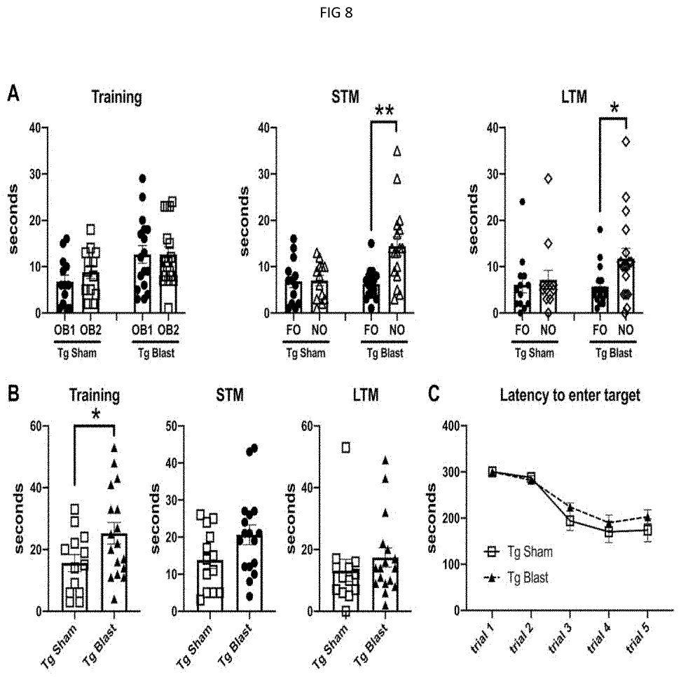

In NOR (FIG. 8A), blast-exposed APP/PS1 Tg mice spent more time exploring the NO in both the STM and LTM testing while the sham exposed APP/PS1 Tg explored the NO and FO a similar amount of time. Blast-exposed APP/PS1 Tg mice also spent more total time exploring the objects during the NOR training session (FIG. 8B). Barnes maze testing (FIG. 8C), revealed that both sham- and blast-exposed APP/PS1 Tg mice showed decreased latencies to enter the target across trials indicating both groups learned the task. However, there were no differences in the learning curve latencies between the sham- and blast-exposed APP/PS1 Tg mice.

When fear learning was tested neither sham- nor blast-exposed APP/PS1 Tg mice showed increased freezing after the presentation of the tone/shock pairings during the training session suggesting that neither group responded normally to the US. In the cued phase testing, neither sham- nor blast-exposed APP/PS1 Tg mice responded with freezing after presentation of the tone consistent with neither group having formed an association between of the tone with the shock during the training session.

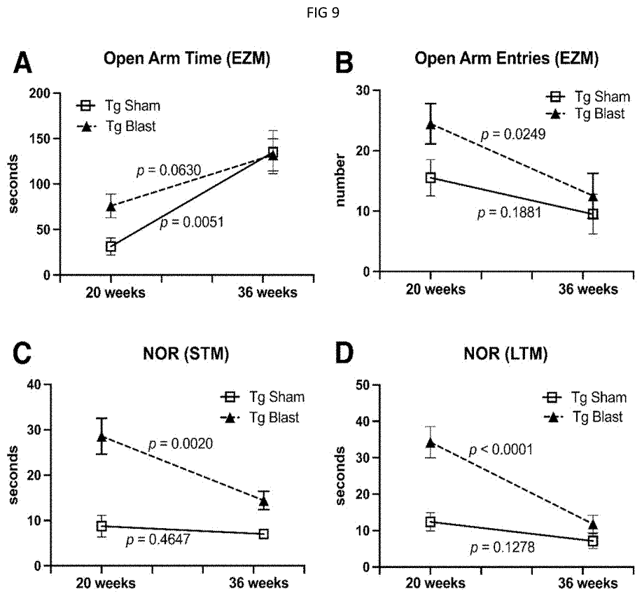

Thus, repetitive low-level blast exposure was less successful at improving behavior in APP/PS1 Tg mice when begun at 36 weeks of age than at 20 weeks of age. Exposure beginning at 36 weeks did not improve Barnes maze performance or rescue fear learning. It also did not improve social interactions. While improving performance in NOR, and partially improving anxiety measures, it appeared less effective at 36 weeks, e.g. not improving open arm time in the EZM (FIG. 7), which was improved at 20 weeks (FIG. 3). To further explicitly test the effect of age at time of exposure we performed simple linear regressions comparing behavioral parameters at 20 weeks to 36 weeks. As shown in FIG. 9A, although open arm time increased in Tg Blast and Tg Sham between 20 weeks and 36 weeks, the increase in Tg Blast did not reach statistical significance while the increase in Tg Sham was statistically significant. By contrast, open arm entries in Tg Blast significantly decreased between 20 and 36 weeks but did not change in Tg Sham (FIG. 98). In NOR, Tg Blast spent less time exploring the NO at 36 weeks compared to 20 weeks in both STM and LTM testing while NO exploration time was not significantly different in Tg Sham (FIGS. 9C and 9D). Thus, in both tests, the diminished effect of blast exposure at 36 week reflected a worsening of performance in Tg Blast rather than a change in performance of Tg Sham. This failure may reflect the more advanced amyloid pathology present in APP/PS1 Tg mice at 36 weeks of age³⁹, which rendered them less responsive to the effects of blast exposure.

Repetitive low-level blast exposure initiated at 20 weeks of age returns many behavioral parameters in APP/PS1 Tg mice to the levels of non-transgenc wild type mice.

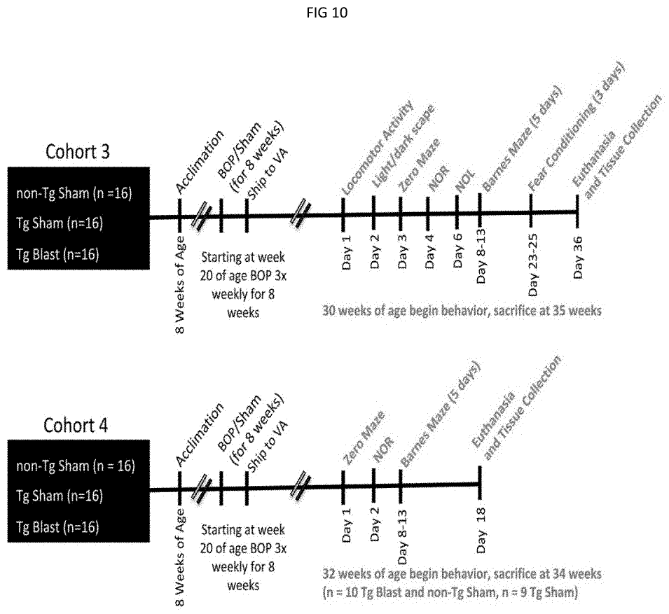

To determine whether repetitive low-level blast exposure could return behavioral parameters in APP/PS1 Tg mice to the levels of non-transgenic wild type mice, we repeated experiments utilizing two additional cohorts of mice (cohorts 3 and 4) that included a control group consisting of sham-exposed non-transgenic (non-Tg) littermates. The three groups (non-Tg Sham, Tg Sham and Tg Blast) received three blast exposures per week for 8 weeks beginning at 20 weeks of age. The groups and group sizes are indicated in Table 1. The timing of behavioral testing and tissue harvesting is shown in FIG. 10. Results for behavioral testing of cohorts 3 and 4 are summarized in Table 1.

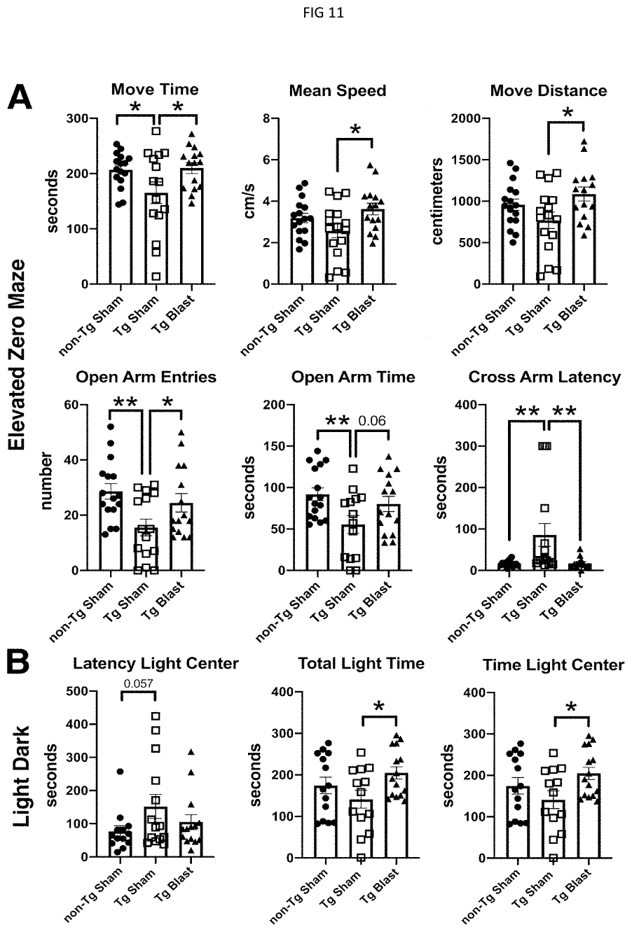

FIG. 11 shows testing of cohort 3 in an EZM and a light/dark escape task. Comparing Tg sham to non-Tg Sham in the EZM (FIG. 11A), Tg Sham mice showed evidence of anxiety, moving less and making fewer open arm entries, as well as spending less time in the open arms and exhibiting a prolonged cross-arm latency compared to sham-exposed non-Tg mice. These deficits were rescued in blast-exposed APP/PS1 Tg mice with all parameters in Tg-Blast mice being similar to sham-exposed non-Tg controls. Similar trends were found in the light/dark escape task (FIG. 11B). While total time spent on the light side and total time in the light center was reduced in Tg Sham compared to Tg Blast, Tg Blast and non-Tg Sham did not differ. Thus, repetitive low-level blast exposure rescued the anxiety phenotype found in sham-exposed APP/PS1 Tg mice.

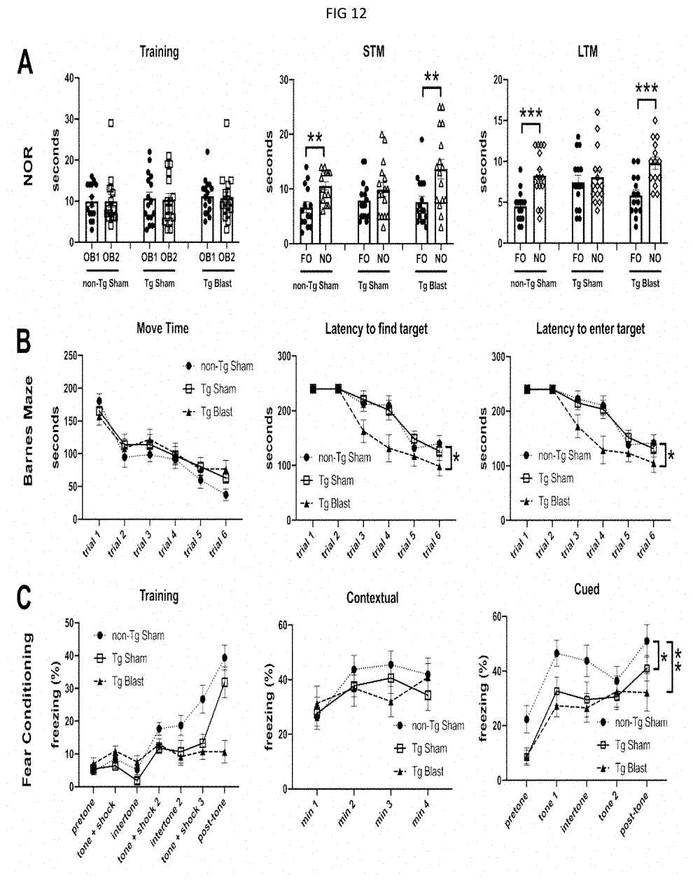

Testing in a NOR task is shown in FIG. 12A. In both STM and LTM testing, sham-exposed Tg mice failed to distinguish the FO and NO. By contrast, sham-exposed non-Tg and blast-exposed Tg mice spent more time exploring the NO than the FO in both STM and LTM testing. Thus, blast exposure rescued recognition memory deficits in APP/PS1 Tg mice. In a Barnes maze, all three groups learned the task, exhibiting progressively shorter latencies across trials to enter the target quadrant or the escape hole (FIG. 12B). However, blast-exposed Tg mice exhibited shorter latencies both to enter the target quadrant as well as enter the escape hole compared to either the non-Tg Sham or Tg Sham groups.

Interpretation of the fear conditioning results for cohort 3 (FIG. 12C) was complicated by the fact that blast-exposed APP/PS1 Tg mice did not show increased freezing across the training trials unlike the wild type non-Tg Sham and APP/PS1 Tg Sham groups suggesting that Tg Blast mice at baseline exhibited abnormal freezing behavior. In the contextual phase testing, freezing in Tg Blast mice was similar to the other two groups suggesting that Tg Blast mice nevertheless had intact memory for the context in which the shocks were presented. In the cued phase testing, when pre-tone freezing was compared to the first presentation of the tone, all groups showed increased freezing. However, Tg Sham and Tg Blast froze significantly less than non-Tg Sham mice (FIG. 12C). Comparing freezing across all trials gave similar results revealing that Tg Sham and Tg Blast mice froze significantly less than non-Tg sham mice (FIG. 12C).

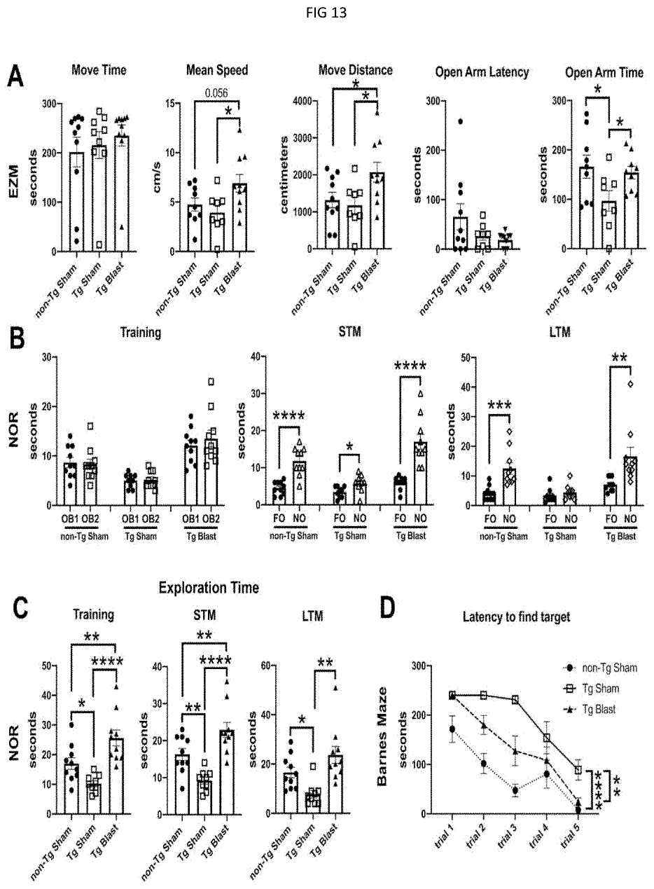

FIG. 13 shows testing of cohort 4 in and EZM and NOR. Comparing Tg Sham to non-Tg Sham in the EZM (FIG. 13A), Tg Sham mice showed evidence of anxiety, moving less distance and spending less time in the open arms compared to sham-exposed non-Tg mice. These deficits were rescued in blast-exposed APP/PS1 Tg mice with parameters being restored to sham-exposed non-Tg controls. FIG. 13B shows testing in a NOR task. In LTM testing, sham-exposed Tg mice failed to distinguish the FO and NO. By contrast, sham-exposed non-Tg and blast-exposed Tg mice spent more time exploring the NO than the FO in LTM testing. Sham-exposed Tg mice spent less total time exploring the objects in all three sessions compared to non-Tg sham mice (FIG. 13C). This effect was rescued in blast-exposed Tg mice that spent more time exploring the objects than non-Tg Sham mice in training and STM testing. In the Barnes maze (FIG. 13D), non-Tg sham mice and blast-exposed Tg mice learned to find the target significantly faster than Tg Sham mice although the learning curves of the Tg blast mice were not as sharp as those of the non-Tg Sham mice. Thus, blast exposure rescued anxiety and recognition memory deficits in sham-exposed APP/PS1 Tg mice and improved spatial memory compared to sham-exposed APP/PS1 Tg mice. Table 1 summarizes the behavioral testing results in cohorts 3 and 4.

Repetitive low-level blast exposure reduces soluble, insoluble, and oligomerc Aβ levels, but amyloid plaque burden is unchanged by blast exposure.

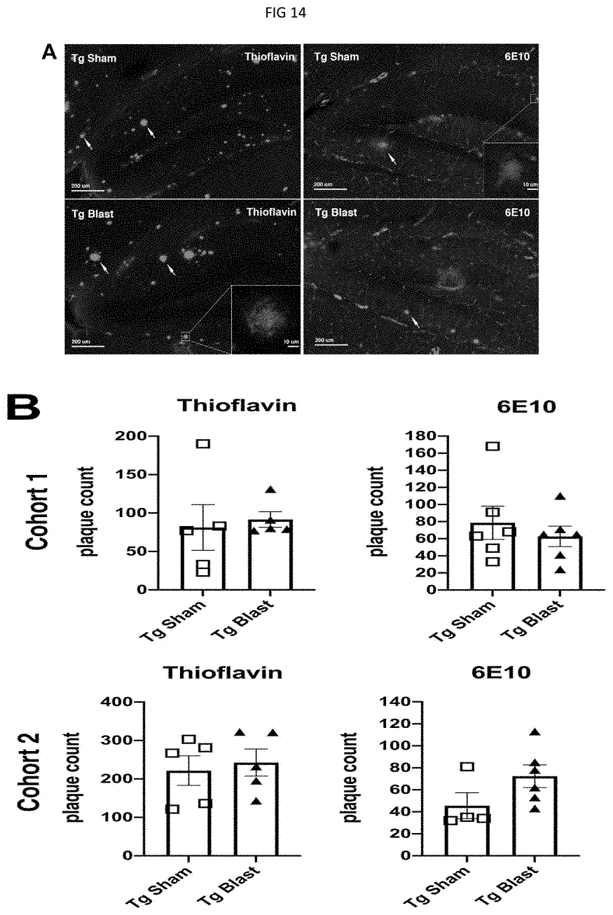

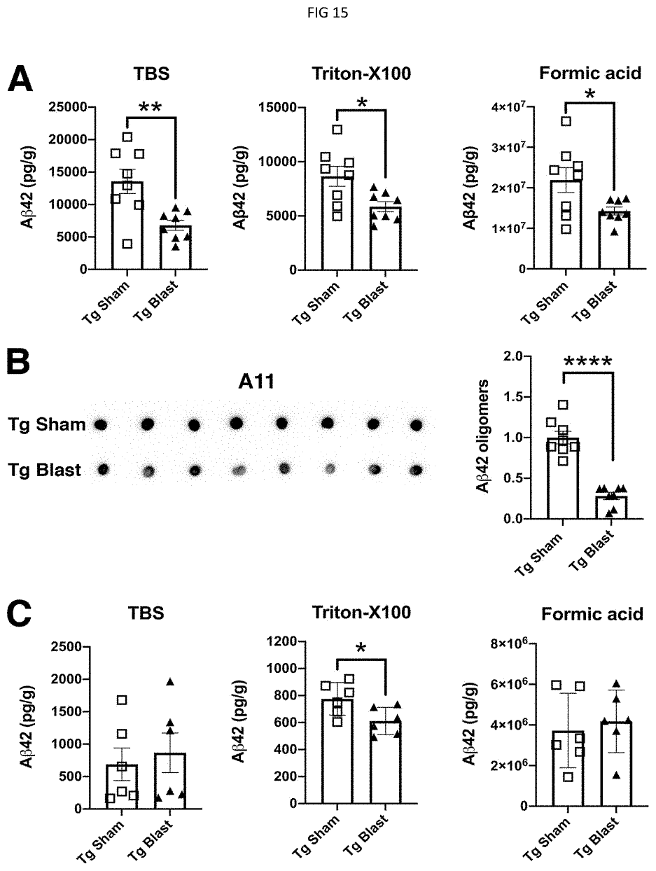

To determine the effects of repetitive low-level blast exposure on plaque load, we measured plaque loads in APP/PS1 Tg mice from cohorts 1 and 2 subjected to blast or sham conditions. Using either thioflavin S staining or immunohistochemical staining with the antibody 6E10, plaque counts were unchanged in these mice (FIG. 14). We next examined Aβ42 levels in brain of APP/PS1 Tg mice from cohort 3 by ELISA using tissue collected after behavioral testing which finished when mice were approximately 9 months of age (7 weeks after the last blast exposure; FIG. 8). Aβ42 was decreased in TBS, Triton X-100, and formic acid-extractable fractions in blast-compared to sham-exposed mice (FIG. 15A). Levels of oligomeric Ab were determined in cohort 3 using monoclonal antibody A11. As shown in FIG. 15B, oligomeric Ab in blast-exposed APP/PS1 Tg mice was decreased to about 33% of that in sham-exposed APP/PS1 Tg mice. Additionally, we examined Aβ42 in a group of mice from cohort 4 that were euthanized within one week of the last blast exposure. In these mice, which were euthanized at 6 months of age and thus younger than cohort 3, Aβ42 was decreased in the Triton X-100 fraction while Aβ42 in TBS and formic acid-extractable fractions were unchanged (FIG. 15C). These studies thus show that while repetitive low-level blast exposure does not alter amyloid plaque load, Aβ42 levels and Ab oligomers are reduced and these reductions are sustained for at least 3 months following the last blast exposure.

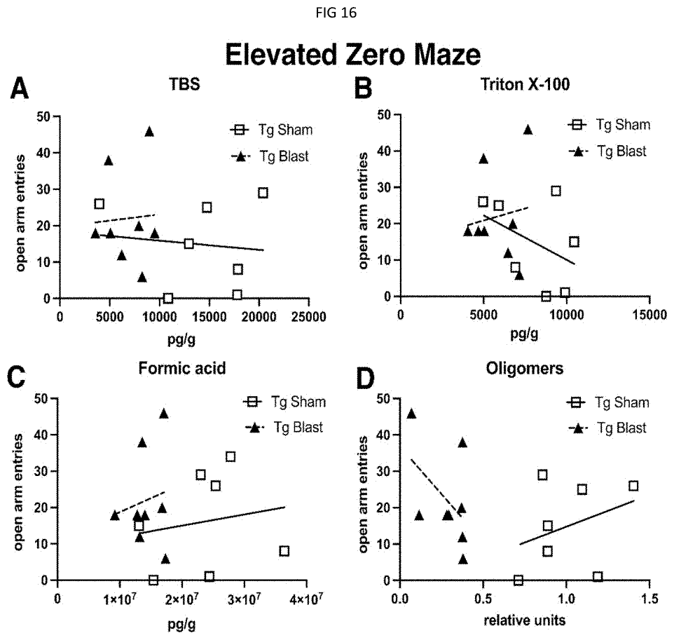

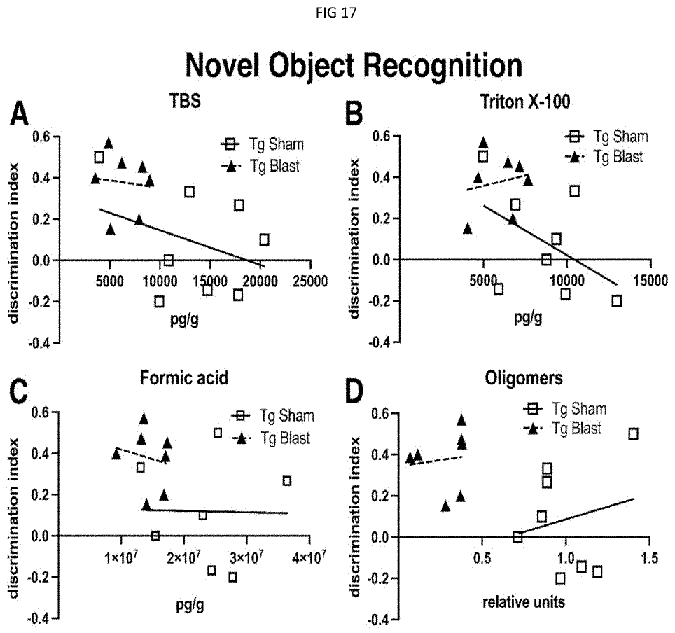

Next, we determined whether levels of soluble, insoluble or oligomeric Aβ42 could be directly correlated with behavioral parameters in individual animals in cohort 3. Table 2 shows correlation coefficients calculated between Aβ42 levels and open arm entries in the EZM or the DI in novel object recognition. There were no significant correlations when the blast or sham were analyzed separately and only one significant negative correlation between DI and TBS soluble Aβ42 when the sham and blast were pooled (Table 2). FIG. 16 shows open arm entries in the EZM correlated with Aβ42 levels. There was a relatively tight clustering of Aβ42 levels in all of the fractions in the Tg Blast, although no correlation was apparent between Aβ42 levels and number of open arm entries in individual animals. While data was generally more spread in Tg Sham, there was again no correlation between Aβ42 levels and number of open arm entries in individual animals Relatively similar results were seen when a DI was calculated for cohort 3 in the STM testing of NOR and correlated with levels of Aβ42 in individual animals (FIG. 17). Thus, while soluble, insoluble and oligomeric Aβ42 correlate with behavioral parameters in the aggregate, they did not correlate with behavioral performance in individual animals.

TABLE 2
Correlation between Ab42 levels and behavioral parameters
in the elevated zero maze and novel object recognition.

| | Pearson | | Kendall's tau-b | | Spearman's rho | |
|---|---|---|---|---|---|---|
| | Correlation | p | Correlation | p | Correlation | p |
| | coefficient | value | coefficient | value | coefficient | value |
| Open arm entries (EZM) | | | | | | |
| Sham: | | | | | | |
| TBS | −.237 | .572 | −.071 | .805 | −.167 | .693 |
| Triton X-100 | .070 | .869 | .071 | .805 | .143 | .736 |
| Formic acid | .006 | .989 | .143 | .621 | .190 | .651 |
| Oligomeric | .260 | .534 | .214 | .458 | .238 | .570 |
| Blast: | | | | | | |
| TBS | .056 | .896 | −.038 | .899 | .000 | 1.000 |
| Triton X-100 | .132 | .756 | .038 | .899 | .098 | .818 |
| Formic acid | .157 | .710 | .113 | .702 | .098 | .818 |
| Oligomeric | −.489 | .219 | −.385 | .200 | −.528 | .179 |
| Sham + Blast: | | | | | | |
| TBS | −.227 | .397 | −.160 | .391 | −.233 | .385 |
| Triton X-100 | −.042 | .877 | −.127 | .498 | −.147 | .586 |
| Formic acid | −0.70 | .798 | .008 | .964 | .024 | .931 |
| Oligomeric | −.176 | .514 | −.119 | .527 | −.151 | .578 |
| Discrimination index | | | | | | |
| (NOR) | | | | | | |
| Sham: | | | | | | |
| TBS | −.344 | .404 | −.071 | .806 | −.071 | .867 |
| Triton X-100 | −.486 | .222 | −.357 | .216 | −.476 | .233 |
| Formic acid | .185 | .662 | .000 | 1.000 | .000 | 1.000 |
| Oligomeric | .206 | .625 | .071 | .805 | −.024 | .955 |
| Blast: | | | | | | |
| TBS | −.455 | .258 | −.357 | .216 | −.476 | .233 |
| Triton X-100 | .306 | .461 | .071 | .805 | .119 | .779 |
| Formic acid | .063 | .881 | .143 | .621 | .048 | .621 |
| Oligomeric | .038 | .929 | .327 | .262 | .539 | .168 |
| Sham + Blast: | | | | | | |
| TBS | −.530 | .035 | −.460 | .013 | −.648 | .007 |
| Triton X-100 | −.428 | .098 | −.310 | .095 | −.446 | .083 |
| Formic acid | −.127 | .639 | −.059 | .752 | −.116 | .668 |
| Oligomeric | −.345 | .190 | −.185 | .321 | −.268 | .316 |

Ab42 levels in APP/PS1 Tg mice from cohort 3 in the TBS, Triton X-100 and formic acid fractions as well as levels of A11 reactive Ab42 oligomers were correlated with open arm entries in the EZM and the discrimination index in NOR. Correlations with p values less than 0.05 are indicated in bold. Data is shown as sham (n = 8) or blast (n = 8) analyzed alone or pooled (n = 16; sham + blast).

### Discussion

TBI is a risk factor for later development of neurodegenerative diseases that may have varied underlying pathologies. Aβ deposition is a hallmark of AD and epidemiological studies support an association of severe TBI with later development of AD. Changes in brain Aβ levels occur rapidly after TBI with increased levels of soluble Aβ and diffuse cortical deposits present in humans as early as two hours after a severe injury. Aβ elevations also occur acutely in brain in many experimental animal models that mimic the type of contusional and rotation/acceleration injuries associated, for example, with motor vehicle accidents or sports injuries. In these models, there is also increased expression of APP, along with BACE1 (b-site APP cleaving enzyme 1), the principal b-secretase and the g-secretase complex that together are responsible for generating Aβ. It has been suggested that upregulation of this amyloidogenic APP processing pathway which favors Aβ production over other non-amyloidogenic APP processing pathways may help explain the epidemiological associations between TBI and AD.

We were thus surprised in a previous study that in both rat and mouse models of blast exposure rather than being increased, rodent brain Aβ42 levels were decreased following acute exposure. Here we subjected a transgenic mouse model of AD to an extended sequence of repetitive low-level blast exposures designed to mimic the equivalent of a human subclinical blast exposure around 5 psi that do not present acute symptoms. Because blast-related brain injury may involve a combination of injuries related to effects of the primary blast wave as well as damage from rotation/acceleration injury, during the blast overpressure exposures head motion is restricted to minimize rotation/acceleration forces. Studies using this exposure level (34.5 kPa) in rodents produce no obvious neuropathological effects or acute behavioral deficits. Because multiple subclinical blast exposures are common for many service members in combat as well as non-combat settings, we utilized a protocol that involved three exposures per week delivered one exposure per day over an 8-week period. We began exposures at 20 or 36 weeks of age, which respectively represent times before or after this line of APP/PS1 Tg mice develop significant plaque burdens.

The inventors show that repetitive blast exposures improved behavioral deficits (Table 2) and chronically lowered Aβ42 in brain. Improved behavioral effects were seen across a range of anxiety related tests (EZM, light/dark, open field). Improved cognition was seen in NOR and NOL tasks as well as Barnes maze. Blast exposure also improved social behavior. These effects were most apparent in APP/PS1 Tg mice that received blast exposures beginning at 20 weeks of age. Beneficial effects were not apparent only in fear learning. Results were less robust in mice when blast exposure began at 36 weeks of age, likely reflecting the greater difficulty of reversing behavioral deficits in mice with more extensive amyloid burden. When these experiments were repeated with inclusion of sham exposed non-Tg littermates, repetitive low-level blast exposure returned many behavioral parameters in APP/PS1 Tg mice to the levels of non-Tg wild type mice.

Accompanying improved behavior, soluble, insoluble, and oligomeric Aβ42 levels were reduced in brain of mice exposed to repetitive low-level blast exposure. This was most apparent in brain of APP/PS1 Tg mice from cohort 3 in which tissue was collected after behavioral testing that finished when mice were about 9 months of age. In these mice, Aβ42 was decreased in TBS, Triton X-100, and formic acid-extractable fractions in blast-compared to sham-exposed mice. Furthermore, Aβ oligomers in cohort 3 were decreased to approximately 33% of the levels in sham-exposed APP/PS1 Tg mice. Oligomeric Aβ is generally considered the most toxic Aβ species. Its lowering following blast exposure is consistent with this being one mechanism of blast's beneficial effect. However, while behavior in the aggregate improved in blast-exposed mice, there was no correlation between oligomeric Aβ or Aβ42 levels in any of the fractions measured with behavioral parameters in individual animals suggesting that other factors are influencing behavioral outcomes as well.

Aβ42 was also determined in a group of mice from cohort 4 that were euthanized within one week after the last blast exposure. In these mice, which were euthanized at 6 months of age and thus younger than cohort 3, Aβ42 was decreased in the Triton X-100 fraction, while Aβ42 in TBS and formic acid-extractable fractions were unchanged. Interestingly, despite the changes in Aβ42 levels, amyloid plaque burdens were unchanged in APP/PS1 Tg mice whether the blast exposure protocol began at 20 weeks (cohort 1) or 36 weeks (cohort 2) of age. Therefore, while repetitive low level blast exposure does not alter amyloid plaque load, Aβ42 levels and Aβ oligomers were reduced and these reductions are sustained for at least 3 months following the last blast exposure.

One previous study examined the effect of blast injury on the same APP/PS1 Tg mouse line studied here. In this study, which focused primarily on retinal injury, APP/PS1 Tg mice were exposed to a single 20-psi (137.9-kPa) blast exposure at 2 to 3 months of age. Two months later, retinal ganglion cell structure and function was impaired in Tg mice compared to non-Tg littermates. No Aβ deposits were detected in retinas of APP/PS1 Tg mice. However, increased APP and Aβ immunoreactivity was found in the blast-exposed Tg animals particularly near blood vessels. In brain, a statistically non-significant trend for greater cortical Aβ plaque load was seen in transgenic blast vs. sham groups (Harper et al). This study differs from ours in both the relatively high level of blast exposure and time-course of studies suggesting that differences in blast dose and frequency may engage different targets after injury. Another recent study also found that normally regulated transgenic overexpression of wild type human APP does not contribute to deficits acutely after TBI and may in fact be protective (Maigler et al). Thus effects may be complex and at least partly related to the presence of the FAD related mutations in the transgene.

Studies in U.S. military personnel have documented the relevance of these animal findings to humans by showing that during a 10-day training exercise which involved repeated blast exposure, Aβ42 was lowered in blood at 24 h following blast exposure. Transient reductions in APP and alterations of the APP signaling network in blood were also observed during training exercises that involved a moderate blast exposure. These studies suggest that as in experimental animals, altered APP processing is an effect of acute blast injury although one recent study found elevated serum Aβ42 in military personnel who experienced repeated blast exposures from firing 0.50-caliber rifles in training sessions conducted over multiple days. Thus, effects in humans may vary with the type and intensity of exposure.

Our current findings do not explain why Aβ is decreased by repetitive low-level blast exposure. Nonetheless, it is notable that Aβ enzymatic production, proteolysis, and transport out of the brain are regulated by multiple, sometimes competing, processing pathways that can be stimulated and/or suppressed by mild traumatic insults to the brain. For example, Aβ can be internalized and degraded by microglia. There is evidence that a mild blast stimulates microglia to migrate toward and internalize substances that have aberrantly crossed the blood-brain barrier (BBB) presumably a neuroprotective response attempting to restore normal BBB functions. The ability of the very mild CNS injuries produced by the low-level subconcussive blasts our animals were exposed to could plausibly be expected to favor activating some neuroprotective pathways that could facilitate reducing Aβ, which in our transgenic mice is otherwise pathogenic. Similarly, Aβ can be cleared from brain by a number of distinct proteolytic pathways. Moreover, there is growing evidence that transport across the BBB, as well as by astroglia-mediated interstitial fluid bulk flow through the perivascular glymphatic system conduct substances, including Aβ and tau into the perineural sheaths of cranial and spinal nerves, meningeal lymphatic vessels and arachnoid granulations. A pathway that drains along the olfactory nerve through the cribriform plate has also been described SS.

In previous studies we found that as in non-blast models, levels of APP were increased following blast exposure although there was no evidence of axonal pathology based on APP immunohistochemical staining. However, unlike findings in non-blast TBI animal models, levels of the BACE-1, and the g-secretase component, presenilin-1 were unchanged following blast exposure. Thus, lowered enzymatic processing of APP seems unlikely to explain the current results. Glymphatic flow is reduced prior to the appearance of substantial amyloid plaque burden in the same APP/PS1 Tg mouse line we used. Consistent with a role for glymphatic flow in the amyloid pathology of APP/PS1 Tg mice, deep cervical lymph node ligation has been reported to exacerbate amyloid pathology, while treatment with a compound that promotes perivascular Aβ drainage improved cognitive performance as well as reduced parenchymal Aβ levels and plaque deposition. Vascular disease, which is prominent after blast-related TBI, may also impair glymphatic outflow after TBI. How glymphatic transport is affected by a low-level repetitive blast exposure and whether more intense blast exposures could affect this brain clearance system differently than low-level blasts is however not fully understood.

### CONCLUSION

Future studies are needed to elucidate the mechanism(s) for how repetitive blast exposure improves behavioral performance and reduces Aβ levels. Such investigations will have practical implications for the treatment of acute blast injury, as blocking Aβ production by pharmacological or genetic means has been reported to reduce tissue damage acutely and improve outcome following controlled cortical impact injuries in mice. However, the studies reported here, together with our previous findings following acute blast exposure, suggest that such strategies may not be applicable to treatment of chronic blast injury if Aβ is already lowered. Rather these findings suggest that paradoxically low-level repetitive blast exposure might actually be beneficial for AD-related cognitive and behavioral changes.

### Example 2: Exposure to Low-Intensity Blast Increases Clearance of Brain Aβ

### Materials and Methods

The animal study protocol was reviewed and approved by the Walter Reed Army Institute of Research (WRAIR)/Naval Medical Research Center (NMRC) Institutional Animal Care and Use Committee in compliance with all applicable Federal regulations governing the protection of animals in research. The experiments reported herein were conducted in compliance with the Animal Welfare Act and per the principles set forth in the “Guide for Care and Use of Laboratory Animals,” Institute of Laboratory Animals Resources, National Research Council, National Academy Press, 2011. Male Long-Evans hooded rats (300-350 g, 10-12 weeks old at study initiation; Charles River, Mass.) were used for the study. Animals were pair-housed with a 12 h light/dark cycle and had ad libitum access to food and water.

### Experimental Blast Overpressure Exposure

Rats were subjected to a single BOP exposure in an air-driven shock tube in the facing orientation, as previously described (Ahlers et al., 2012). The tube is a 19.5 ft. long, with a 12-inch circular diameter. The shock tube is divided into a 2.5 ft. compression chamber that is separated from a 17 ft. expansion chamber by polyethylene MYLAR™ sheets (Du Pont Co., Wilmington, Del., USA) that control the generated peak pressure of the blast wave. The peak pressure at the end of the expansion chamber was determined by piezoresistive gauges specifically designed for pressure-time (impulse) measurements (Model 102M152, PCB, Piezotronics, Inc., Depew, N.Y., USA). Prior to blast exposure, rats were anesthetized with 4% isoflurane for 3 minutes. To restrain movement of the head and body, rats were placed in a plastic Decapi Cone (Braintree, Inc) and secured into a metal holding basket inside the shock tube using three tourniquets attached to the metal basket. Rats received the equivalent of 37 kPa (˜5.4 psi) blast overpressure (peak pressure: 6.40±0.12 psi (mean±SD); impulse 1.38e-2±3.5e-4 psi*s (mean±SD)). Sham (control) animals were exposed to all procedures, including anesthesia, restraint, and placement inside the shock tube, except for exposure to blast overpressure.

Due to the differences in assay requirements regarding tissue processing, separate groups of animals were used for enzyme-linked immunosorbent assay (ELISA) and electrochemiluminscent multiplex assays (n=15 rats/group), Western blotting (n=6 rats/group), and immunohistochemistry (n=6 rats/group).

### Biosamples

At 24 hours (1 d) or 28 days (28 d) after exposure to BOP or sham procedures, animals were administered sodium pentobarbital (150 mg/kg) for euthanasia. CSF was collected through a cannula implanted in the cisterna magna. Rat CSF was collected in polypropylene tubes, centrifuged at 1200×g for 10 minutes at 4° C. and immediately stored at −80° C. until assay. Blood was collected by intracardiac puncture into ethylenediaminetetraacetic acid (EDTA) tubes and centrifuged at 1000×g for 15 minutes at 4° C. Plasma samples were aliquoted in polypropylene tubes and stored at −80° C. until assay. After CSF collections brains were extracted and immediately frozen on dry ice.

### ELISA

Frozen brains were prepared for ELISA as previously described (Steele et al., 2009; De Gasperi et al., 2012). Whole brains stored at −80° C. were thawed and the cerebral cortex was collected using a brain-trimming matrix. Cortical tissue was homogenized on ice using a Dounce glass hand-held homogenizer. Phosphate buffered saline (PBS)-soluble fractions were extracted first using a buffer containing 1% 5 mM EDTA and 1 X protease and phosphatase inhibitor cocktail in PBS. The extracts were centrifuged at 13,000×g for 30 minutes at 4° C. and the supernatant was collected. The remaining insoluble pellet was used to extract Triton X-100 fractions. The pellet was re-suspended in a buffer containing 15% 150 mM NaCl, 5% 50 mM Tris-HCl buffer, pH 8.0, 1% Triton X-100, 1 X protease phosphatase inhibitor cocktail, and 1% 5 mM EDTA. The resulting homogenate was centrifuged at 13,000×g for 30 minutes at 4° C. and the Triton soluble fraction of the samples was collected from the supernatant.

Protein levels in the PBS and Triton X-100 fractions were determined using a Pierce BCA Protein Assay kit (Thermofisher Scientific). Aβ 40 and 42 levels were determined using human/rat Aβ 40 (Wako II, 294-64701, Fujifilm, Japan) and Aβ 42 (Wako, 292-64501, Fujifilm, Japan) ELISA kits following the manufacturer's instructions. For each sample, 100 μg of protein were used to determine Aβ 40 and 42 levels in both the PBS and Triton fractions.

### Oligomeric Aβ Dot Blot Assay

The levels of Aβ oligomers in PBS brain fractions were determined using a dot blot assay and the A11 anti-oligomer antibody (Thermofisher). 2.5 μg of protein of each sample were applied to a nitrocellulose membrane, allowed to dry, washed in tris-buffered saline (TBS), and incubated in a blocking buffer consisting of 5% nonfat milk in TBS for 1 h. The membranes were then washed and incubated in a solution containing A11 in milk at a concentration of 1:2000 for 1 h at room temperature followed by three washes and incubation in HRP anti-rabbit secondary antibody for 1 h. At the end of the incubation period, the membranes were washed and the blots were developed with an enhanced chemiluminescence reagent (SuperSignal WestFemto, Thermo Scientific) and imaged (Amersham 680, Cytivia) to detect the HRP-conjugated antibody complex. Image J was used to quantify blot density.

### Electrochemiluminescent Multiplex Immunoassay for Aβ and Cytokines

Analyses of Aβ 40 and 42 levels in plasma and CSF were determined using an electrochemiluminscent multiplex assay system (V-PLEX Aβ Peptide Panel 1 (4G8) Kit, catalog No. K15199E-1, Meso Scale Discovery (MSD), Rockville, Md.). Plasma and CSF samples were prepared and loaded on the assay plate following the manufacturer's instructions. An electrochemiluminescense (ECL) Meso Scale Discovery platform (MESO QuickPlex SQ 120MM Reader, MSD, MD) was used to detect the plate-captured antigens. The data were analyzed by MSD Discovery Workbench Software (v. 4.0) using curve fit model (4-PL with 1/y\hat{ }2 weighting as the default fit). The choice in using this multiplex assay for detecting Aβ in plasma and CSF is justified in the ability of the assay to detect multiple Aβ peptides in the same sample and allow for sample conservation.

Plasma and brain tissue cytokine levels of tumor necrosis factor alpha (TNF-α) were measured using MSD sensitive V-plex format electrochemiluminescence TNF-α immunoassay kit (MSD, catalogue No. K153QWD-1). A total 200 μg protein in 50 μl samples were loaded and the assay was run following manufacturer's instructions.

### Western Blotting

For assessment of APP and Aβ peptides, protein extracts were prepared from Triton X-100 fractions or PBS fractions as described above. For assessment all other proteins, protein extracts were prepared from cortical tissue, which was lysed on ice in a buffer containing 150 mM NaCl, 50 mM tris-HCl, 0.25% deoxycholate, 1 mM EGTA, 1 mM NaF, 1 mM Na₃VO₄, and a cocktail of proteinase inhibitors. Sample protein concentration was determined using a Pierce BCA assay (Thermofisher). Western blotting was performed as previously described. Briefly, 25 μg of each sample were subjected to SDS-PAGE electrophoresis and transferred to PVDF membranes. The membranes were probed with primary antibodies at 4° C. overnight, with the exception of the antibody against the housekeeping protein β-actin, which was incubated for 45 min at room temperature.

The primary antibodies and antibody concentration utilized in the experiment were as follows: mouse monoclonal anti-Aβ clone M3.2 (1:1000; 805701, Bio Legend, San Diego, Calif.); rabbit anti-BACE1 (1:2000; ab263901, Abcam, Cambridge, Mass.); rabbit anti-PSN1 (1:10000; 5643, Cell Signaling Technologies, Danvers, Mass.); mouse anti-ADAM10 (1:1000; sc-48400, Santa Cruz Biotechnology, Dallas, Tex.); rabbit anti-ADAM17 (1:2000; 703077, Invitrogen, Carlsbad, Calif.); rabbit anti-phospho-ADAM17 (1:1000; PA5-104938, Invitrogen); rabbit anti-AQP4 (1:1000, ab46182, Abcam); mouse anti-claudin 5 (1:1000; 35-2500, Invitrogen); mouse anti-occludin (1:1000; 33-1500, Invitrogen); mouse anti-ZO-1 (1:1000; 33-9100, Invitrogen); rabbit anti-LRP1 (1:20,000; ab92544, Abcam); and mouse anti-O actin (1:10000; A5441, Sigma Aldrich, Saint Louis, Mo.) and rabbit anti-O actin (1:10000; 4967, Cell Signaling). Following incubation with the appropriate HRP-linked IgG secondary antibodies (Cell Signaling), the blots were developed with an enhanced chemiluminescence reagent (SuperSignal WestFemto, Thermo Scientific) and imaged (Amersham 680, Cytivia) to detect the HRP-conjugated antibody complex. Image Quant TL (Version 8.2.0, Cytivia) was used for quantification of blot density. Finally, each membrane was incubated in a stripping buffer for 15 min to remove the immune complex and re-probed for the housekeeping protein β-actin, which was used as a loading control.

### β- and α-Secretase Activity Assays

The activity of β-secretase BACE1 was determined using β-Secretase (BACE1) Activity Detection Kit (Catalog #: CS0010, Sigma-Aldridge, Saint Louis, Mo.). Brain cortical tissue was homogenized in a solution containing 150 mM NaCl, 50 mM Tris-HCl, 0.25% deoxycholate, 1 mM EGTA, 1 mM NaF, 1 mM Na₃VO₄, and a cocktail of proteinase inhibitors. Protein concentration was determined using a BCA assay and 130-160 μg of protein was used to determine BACE1 activity following the manufacturer's protocol. Enzyme activity was standardized to concentration and is reported as pmol/μg. The activity of the α-secretase ADAM17, also known as tumor necrosis-α cleaving enzyme (TACE)) was also determined using a commercially available kit (Sensolyte 520 TACE (α-Secretase) Activity Assay Kit; Catalog #: AS-72085; AnaSpec Inc., San Jose, Calif.). Brain cortex was homogenized in a solution containing the appropriate assay buffer with 0.1% (v/v) Triton-X 100. Protein concentration was determined using a BCA assay and 50 ug of protein of each sample was used following the manufactures instructions to determine the enzyme activity.

### Immunohistochemistry

Brains collected from animals exposed to blast or sham procedures were immediately embedded in optimal cutting temperature compound (Tissue-Tek® OCT™ compound, Sakura Finetek Europe B. V) over dry ice. Cryostat sections (5 μm) from 0.8 mm anterior to- and 4.8 mm posterior to bregma were processed for immunohistochemistry as previously described (Abutarboush et al., 2019). Briefly, tissue sections were washed in PBS and blocked in 2% bovine serum albumin in PBS for 30 min at room temperature. Sections were then incubated with primary antibodies overnight at 4° C. The following day and after several washes in PBS, the sections were incubated with the appropriate cyanine (Cy) 2- or Cy3-conjugated secondary antibodies (1:500; Jackson ImmunoResearch, West Grove, Pa.). Sections were then washed in PBS, dehydrated through a graded series of water-ethanol mixtures, cleared with xylene, mounted, and cover-slipped. Sections were examined with an Olympus AX80 (Olympus) or a confocal microscope (Fluoview FV1200, Olympus). ImagePro Premier version 9.3 was used for quantification of immunofluorescence. The primary antibodies and antibody concentration utilized in the experiment were as follows: rabbit anti AQP-4 (1:1000, Santa Cruz Biotechnologies, Dallas, Tex.); mouse anti GFAP (1:2000, G3893, Sigma-Aldrich); rabbit anti-LRP1 (1:2000; ab92544, Abcam); mouse anti-SMTH (1:2000; Invitrogen, Carlsbad, Calif.).

### Statistical Analyses

For all data collected, an outlier analysis and checks on the assumptions for normality and variance were performed. Outliers were excluded and if the data met the normality assumption, parametric tests were utilized. If the normality assumption were not met, a transformation was attempted. If a transformation was not possible, non-parametric tests were used. In each case, first an omnibus test was conducted (e.g., one-way or two-way analysis of variance (ANOVA) for parametric data and Kruskal-Wallis for non-parametric data), followed by pairwise comparisons. Corrections for multiple comparisons were done using Tukey's or Dunnett's methods and adjusted p-values are reported where applicable.

### Results

Reduction in Aβ after Exposure to Low-Level Blast in the Acute Phase

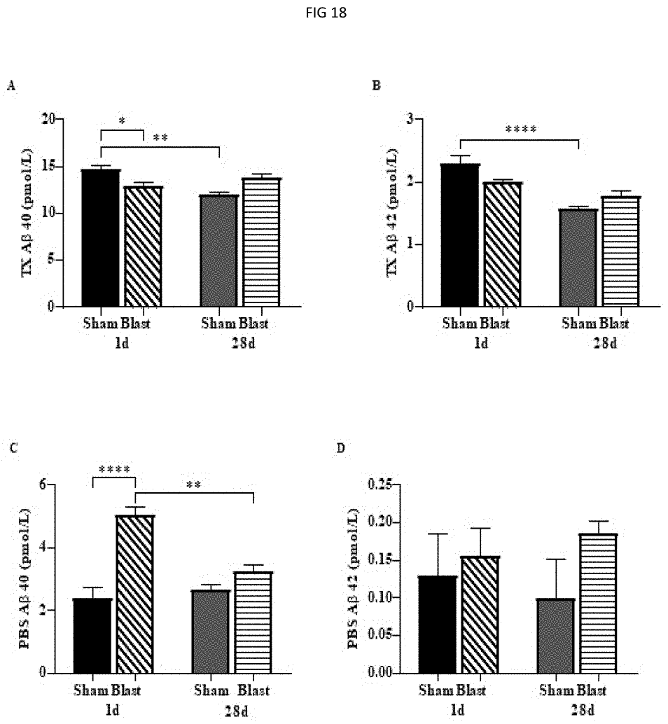

In our model of low-intensity blast exposure in male rats, we quantified the levels of the peptides Aβ 40 and 42 in two different biochemical compartments based on solubility: a detergent (Triton X-100) and a PBS compartment. Similar to our previous findings (De Gasperi et al., 2012), we found higher levels of Aβ 40 and 42 peptides in the Triton X-100 fractions compared to the PBS fractions (FIG. 18). For example, Aβ 40 levels were ˜4× higher in the Triton X-100 fractions in the 28 d sham animals (11.89±0.37 vs. 2.64±0.19 pmol/L in Triton X-100 vs. PBS fractions, respectively). The difference in Aβ 42 levels between the two fractions was larger, with Aβ 42 levels 10-15× higher in Triton X-100 compared to PBS fractions. Overall, PBS fractions had lower signal and high variability. Also consistent with previous rodent and human studies, the levels of Aβ 42 were lower than the levels of Aβ 40 in both Triton X-100 and PBS fractions (Steinerman et al., 2008; De Gasperi et al., 2012; van Etten et al., 2017).

Analyses comparing the levels of Aβ 40 and 42 in Triton X-100 fractions between blast and sham conditions, 1 d (24 h), and 28 d after exposure to blast revealed that exposure to blast results in a reduction in the levels of both Aβ 40 and 42 in the Triton X-100 soluble fractions 1 d after exposure to blast. Specifically, for Aβ 40 (FIG. 18A), a two-way ANOVA using blast exposure and time after injury, showed no significant effects of blast or time but a significant interaction (F=10.57, p=0.0026). Pairwise comparisons revealed a significant reduction in Aβ 40 in blast exposed animals at 1 d compared to sham (Tukey's, p<0.05). In addition, compared to fractions from 1 d sham, there was a significant reduction in Aβ 40 levels in sham animals 28 d after exposure to sham procedures (p<0.05). There were no differences in the levels of Aβ 40 between sham and blast animals at 28 d, in spite of a ˜16% increase in Aβ 40 in blast-exposed animals (11.89 vs 13.76 pmol/L Aβ 40 in sham vs. blast animals at 28 d).

For Aβ 42 Triton X-100 extracts (FIG. 18B), a two-way ANOVA showed a significant effect of time (F=23.33, p<0.0001) and a significant interaction between time and exposure to blast (F=7.34, p=0.011). Pairwise analyses showed that the levels of Aβ 42 in Triton X-100 fractions extracted from brains of animals euthanized 24 h after exposure to blast showed a 13.6% reduction compared to sham animals (Aβ 42=1.98±0.05 pmol/L in 24 h blast-exposed vs.2.29±0.12 pmol/L in 24 h sham fractions). This difference approached but did not reach statistical significance (p=0.057). Although the levels of Aβ 42 increased ˜14% in blast animals 28 days after exposure compared to sham 28 d animals, the difference was not significant. The effect of time on Aβ 42 levels was demonstrated in the unexpected reduction in Aβ 42 levels in sham animals 28 d after exposure to sham procedure compared to sham 24 h animals. Specifically, there was a near 32% reduction in Aβ 42 from 2.29±0.12 pmol/L in sham 24 h Triton extracts to 1.56±0.06 pmol/L in sham 28 d extracts (Tukey's, p<0.0001). In blast exposed animals, brain levels of Aβ 42 in Triton X-100 extracts from animals euthanized 28 d post-blast exhibited a ˜10% decrease compared to those blast-exposed animals euthanized 24 h after exposure, but this difference was not statistically significant. To summarize, exposure to blast is associated with a trend of reduction in brain levels of Aβ 42 at 24 h (but not 28 d) after exposure and time/age appears to be a significant factor in brain levels of detergent-soluble Aβ 42 in sham animals and is associated with a decrease in Aβ 42.

The levels of Aβ 40 and 42 in the PBS extracts showed different trends from those observed in the Triton X-100 fractions. For Aβ 40 (FIG. 18C), a two-way ANOVA showed significant time (F=6.77, p=0.015), exposure (F=30.25, p<0.0001), and interaction (F=12.24, p=0.0017) effects. Pairwise comparisons showed a significant increase in PBS Aβ 40 within 24 h after exposure to blast, where values in blast-exposed brains were more than twice as the sham (2.38±0.36 pmol/L in sham vs 5.04±0.24 pmol/L in blast-exposed brains; Tukey, p<0.0001). There was, however, no difference between sham and blast exposed brains at 28 d after exposure. Notably, Aβ 40 levels were reduced in the 28 d extracts from animals exposed to blast compared to their 1 d counterparts (p=0.001), signifying a significant time effect. Sham animals showed no variation in the levels of PBS Aβ 40 over time. For the PBS Aβ 42 fractions (FIG. 18D), there were no significant differences among the groups, most likely due to the low signal and high variability of the obtained readings.

In summary, our data show that exposure to low-intensity blast is associated with a reduction in brain detergent-soluble (Triton X-100) Aβ 40 and Aβ 42 monomers, 1 d after exposure to blast. This reduction is paralleled by an increase in Aβ 40 in PBS-soluble extracts at 24 h. Time is a factor in the levels of detected brain Aβ 40 and 42. In particular, the levels of Triton X-100 Aβ 40 and 42 decreased significantly in sham animals over time (comparing 24 h to 28 d), while blast-exposed animals showed a reduction in PBS Aβ 40 levels over time.

Plasma Aβ Levels are Reduced after Exposure to Blast

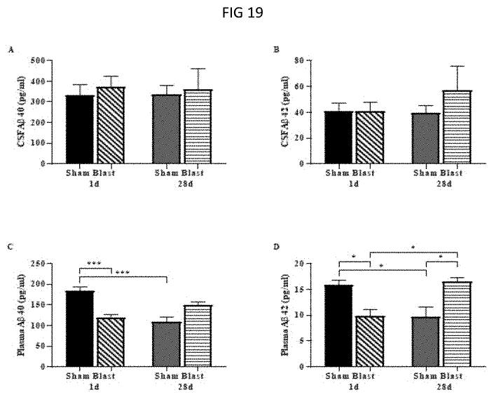

The levels of Aβ 40 and 42 in CSF and plasma were examined using an electrochemiluminescent multiplex assay system and the results are illustrated in FIG. 2. In CSF, there were no significant differences in the levels of Aβ 40 or 42 between CSF from sham and blast animals at 24 h or 28 d after exposure. The levels of Aβ 40 in CSF ranged from 331.9±47.2-370.3±50.3 pg/mL (FIG. 19A), while the levels of CSF Aβ 42 ranged from 39.3±5.1-40.3±6.2 pg/mL (FIG. 19B). In spite of the lack of statistically significant difference in Aβ levels in CSF, there was a ˜8-12% increase in Aβ 40 at 24 h and 28 d. Similarly, there was a 44% increase in CSF Aβ 42 (from 39.4±5.6 to 57.0±18.6 pg/mL), which did not reach statistical significance due to the high variability in the measurements.

For plasma levels of Aβ 40, a two-way ANOVA showed a significant effect of time (F=4.44, p=0.046) and interaction between time and injury (F=25.74, p=<0.0001), with significant differences between 1 d sham and 1 d blast (Tukey, p<0.001) and 1 d sham and 28 d sham (Tukey, p<0.001). Similar to our observations of brain Aβ 40, plasma levels of Aβ 40 exhibited a significant decrease 1 d after blast compared to sham (FIG. 19C). Plasma Aβ 40 levels decreased from 182.3±11.4 pg/ml in sham animals to 117.9±8.9 pg/ml, a 35% reduction. In contrast, plasma Aβ 40 levels increased 28 d post-blast exposure compared to sham, but the difference was not statistically significant. Unexpectedly, sham animals showed a reduction in plasma levels of Aβ 40 over time, with sham 28 d animals having 40% less Aβ 40 than sham 24 h animals.

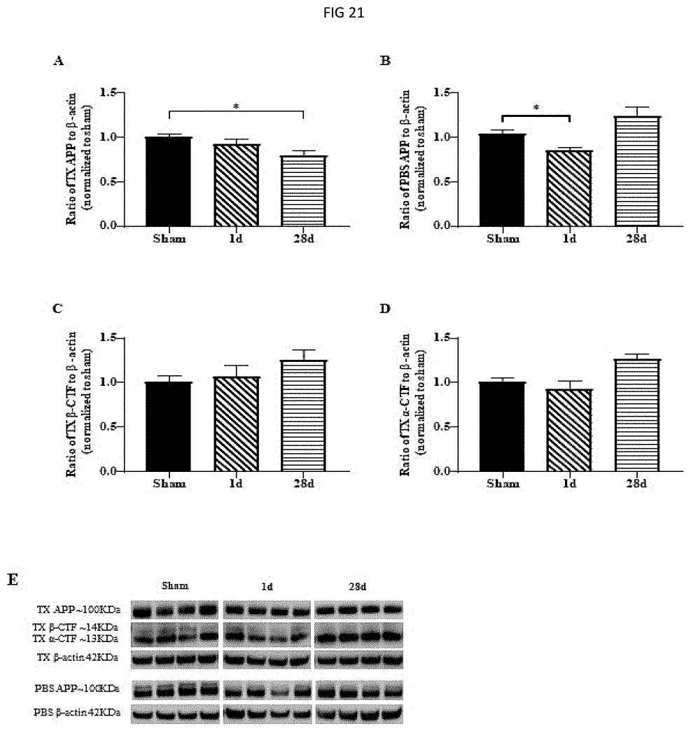

Analysis of plasma Aβ 42 levels using a two-way ANOVA showed a significant interaction effect (F=23.25, p<0.0001), with significant differences between 1 d sham and 1 d blast (Tukey's p=0.012), 24 h sham and 28 d sham (Tukey's p=0.013), 1 d blast and 28 d blast (Tukey's p=0.011), and 28 d sham and 28 d blast (Tukey's p=0.011). In particular, we observed a similar pattern to our observation in brain Aβ 42 levels: a significant reduction in plasma Aβ 42 between 1 d sham and 1 d blast animals (FIG. 21D). The levels of Aβ 42 at 1 d were 15.9±1.0 μg/ml in sham animals and 9.9±1.3 pg/ml in blast-exposed animals. At 28 d, plasma Aβ 42 were 70% higher in blast-exposed animals (9.6±1.9 pg/ml in sham animals and 16.4±1.0 pg/ml in blast-exposed animals). Similar to our observations with Aβ 40, there was a reduction in plasma Aβ 42 levels in sham animals over time, with significantly lower concentrations 28 d after sham procedures (9.6±1.9 pg/ml at 28 d vs 15.9±1.0 pg/ml).

### Oligomeric Aβ Levels are not Affected by Low-Level Blast

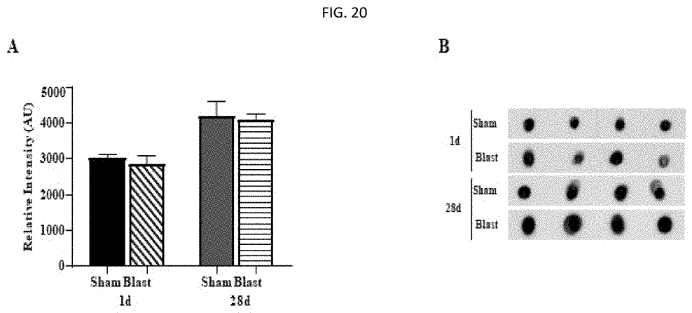

To determine whether the observed reduction in Aβ after exposure to blast is caused by oligomerization of Aβ monomers, we examined the levels of Aβ oligomers in our samples using a dot plot assay. The Triton buffer is incompatible with the antibody used for this assay and therefore only data from the PBS fractions is reported. The PBS-soluble fractions provided robust immunoblots when probed with oligomeric Aβ antibody. The levels of oligomeric Aβ in the PBS-fractions was not different between blast-exposed or sham animals at 1 d or 28 d (FIG. 20A and FIG. 20B). A two-way ANOVA showed that there were no injury or interaction effects, but that there was a highly significant effect of time (F=28.78, p<0.0001).

No Changes in APP and Reduction in Amyloidogenic Species 24 h after Exposure to Blast

APP and Aβ peptides levels in each of the Triton and PBS fractions were also determined using western blotting using the anti-AP clone M3.2 (FIG. 21A-E), which is raised against the rat APP protein and targets a sequence between the 0 and α-secretase cleavage sites. APP full-length protein (˜100 kDa) was detected in both fractions. In addition, the antibody detected two bands between 11-14 kDa in both fractions (FIG. 21E) and a 28 kDa band (not shown) in the PBS fraction. The bands between 11-14 kDa were denser and more conspicuous in the Triton fraction. The ˜14 kDa is believed to correspond to the β-CTF peptide (also referred to as CTFP or C99) and has been reported in brain western blot preparations with the M3.2 antibody by the antibody manufacturer as well as several investigators (Lauritzen et al., 2016; Miranda et al., 2018; Socodato et al., 2020; Tambini et al., 2020). The ˜11 kDa band has been identified by some investigators as α-CTF (also known as C83)(Lauritzen et al., 2016). There were no differences between sham 1 d and sham 28 d values and the data for all sham animals was pooled for analyses.

No differences between sham and blast-exposed animals were observed at 1 d post-blast, but there was a 21% reduction in APP levels in the Triton fractions 28 d (ANOVA F=3.46, p=0.044; followed by Dunnett sham vs. 28 d, p<0.05; FIG. 21A). APP in the PBS fraction showed a 15% decrease compared to sham 1 d post blast (ANOVA F=8.34, p=0.0014; followed by Dunnett, p<0.05 vs. sham; FIG. 218).

β-CTF (C99) is the carboxyl-terminal fragment generated by the amyloidogenic cleavage of APP by β-secretase, while α-CTF (C83) is produced by α-secretase cleavage of the APP molecule. Here we report no statistically significant changes in β-CTF at 1 d or 28 d after exposure to blast (Kruskal-Wallis, p=0.20; FIG. 21C). Similarly, there was an insignificant 26% increase in α-CTF levels 28 d after exposure to blast, without any statistically significant differences among the groups (ANOVA F=2.07, p=0.14; FIG. 21D).

The 28 kDa band has been reported by other investigators in brain lysate preparations probed with this antibody and may correspond to a higher molecular mass product of APP cleavage (Lauritzen et al., 2016) or an oligomeric form of Aβ. There were no difference in the levels of this peptide between the sham and blast-exposed animals at the two time points examined in this study.

### Exposure to Blast Alters APP-Proteolytic Secretase Components

We studied the levels of β, γ-, and α-secretases to determine whether any of the observed changes Aβ levels can be correlated with changes in APP proteolysis. In addition, the activity of the β-secretase BACE1 and the α-secretase ADAM17 (or TACE) were examined here to evaluate whether any of the observed changes in Aβ 40 or 42 could be related to the activity levels of these APP-processing enzymes.

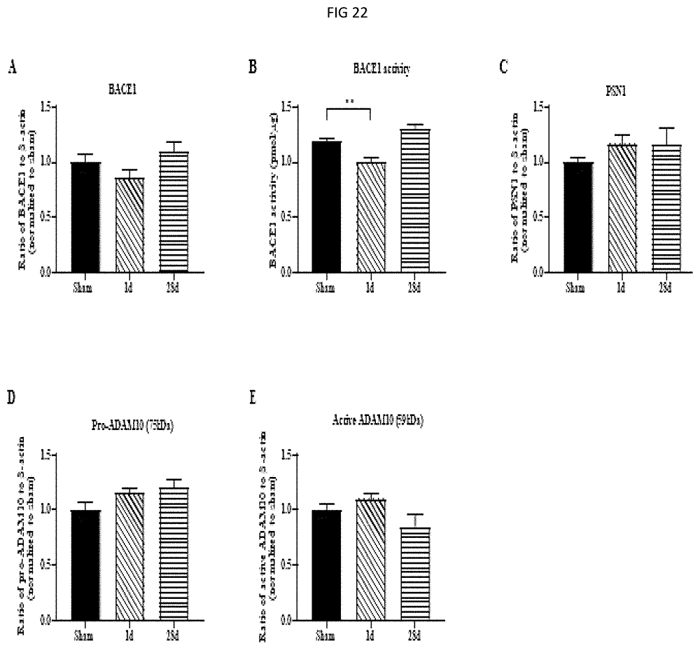

Previous work has shown that non-blast TBI may be related to changes in BACE1 levels. In this study, first western blotting was used to assess the levels of BACE1 in sham and 37 kPa blast-exposed animals. No statistically significant differences were found at 1 d and 28 d after treatment, in spite of a mild 15% reduction in BACE1 24 h after blast exposure compared to the sham group (ANOVA F=2.51, p=0.11; FIG. 22A). However, the mild reduction in BACE1 levels was associated with a significant reduction in the enzymatic activity of the β-secretase (FIG. 22B; Kruskal Wallis p<0.001, followed by Dunn sham vs. 1 d blast groups p=0.0058). This finding may partially explain the observed reduction in both Aβ 40 and 42 at the 1 d time point after exposure to blast. Similar to BACE1 western blotting results, there were no differences in the γ-secretase component PSN1 (Welch's ANOVA W=2.14, p=0.16; FIG. 22C), which cleaves APP following proteolysis with BACE1 to produce Aβ40 and 42.

We also examined the levels of two proteins with α-secretase activity ADAM10 and ADAM17. Two forms of ADAM10 were detected in the brain lysates from our animals: the presumptive immature pro-protein (˜75 kDa) and the mature full length ADAM10 (˜59 kDa) (Tousseyn et al., 2009; Sogorb-Esteve et al., 2018). There were no differences in the levels of either form among the groups (FIG. 22D and FIG. 22E).

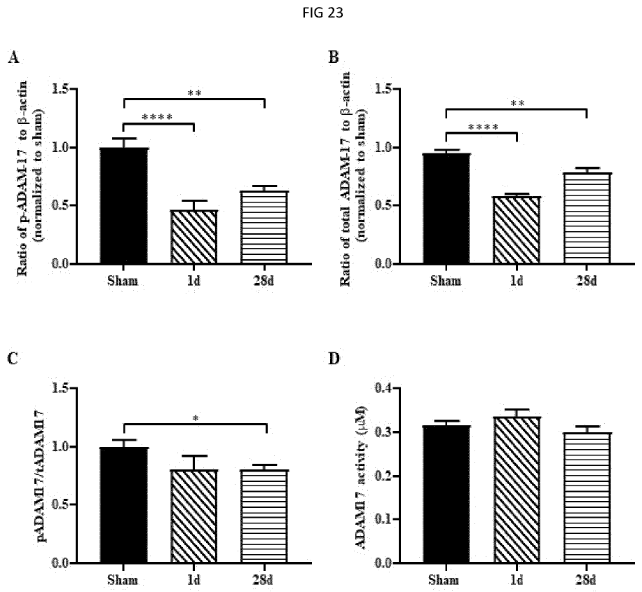

Assessment of ADAM17 by western blotting (FIG. 23) showed a reduction in the levels of phosphorylated and total ADAM17 at both the 1 d and 28 d time points after exposure to blast. ADAM17 phosphorylation at Thr735 was also significantly altered (ANOVA F=16.59, p<0.0001) with a 53% and 37% reduction compared to sham at 1 d and 28 d (Tukey, p<0.05 in each case compared to sham), respectively (FIG. 23A). There was a 42% reduction (ANOVA F=43.41, p<0.0001) in the expression of total ADAM17 within 1 d, which partially recovered to a 21% reduction relative to sham levels at 28 d post-blast (Tukey p<0.05 in each case; FIG. 23B). Assessing the ratio of phosphorylated to total ADAM17 is an approach that can shed some light on changes in the activity of the protein. The ratio of phosphorylated ADAM17 to total ADAM17 was slightly lower in the 28 d group (FIG. 23C), possibly indicating an increase in ADAM17 phosphorylation at that time point. In addition, the activity of ADAM17, assessed using a TACE assay, was not statistically different among the groups in spite of a non-significant reduction in TACE activity in blast-exposed relative to sham animals 24 h after blast (FIG. 23D). How these observations of the ADAM17 α-secretase levels and activity may relate to the observed reduction in Aβ is not clear and some possibilities are addressed in the discussion.

### Changes in Cytokines and Pro-Inflammatory Factors

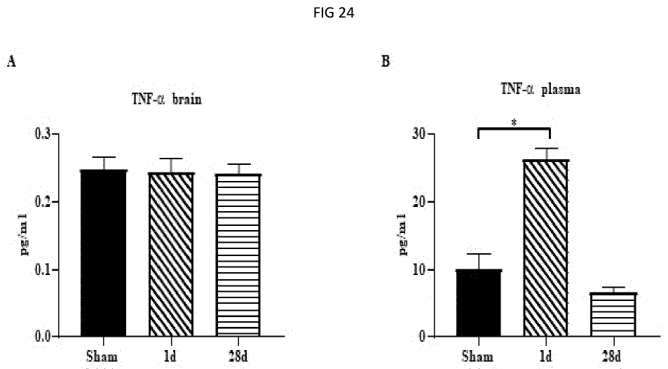

The α-secretase ADAM17 plays an important role in the production of pro-inflammatory cytokines, most notably TNF-α and IL-6 receptor production. The bidirectional interactions of inflammation and Aβ biology in TBI and neurodegenerative diseases including AD (Montgomery and Bowers, 2012), piqued an interest in investigating the relationship between the blast-induced reduction in ADAM17 following exposure to low-intensity blast exposure on TNF-α. In spite of the significant decrease of ADAM17 at the 1 d time point following blast in the brain, a statistically non-significant 8-9% decrease in TNF-α was observed at 1 d and 28 d post-blast (FIG. 24A). Conversely, plasma levels of TNF-α tripled 1 d after low-level blast (Kruskal-Wallis p=0.0006 followed by Dunn's, p<0.05 1 d post-blast vs sham; FIG. 24B). While this increase in peripheral TNF-α is most likely not related to changes in brain levels of ADAM17, it may nevertheless affect Aβ dynamics in the brain and some possibilities are explored in the discussion.

### Exposure to Low-Intensity Blast Alters BBB Vascular Components

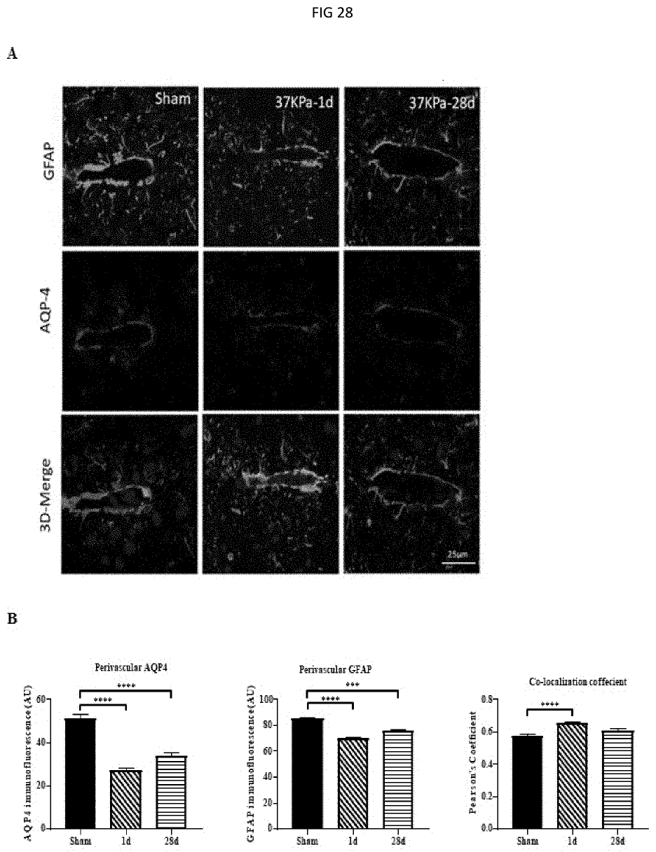

To determine whether the observed blast-induced changes in brain levels of Aβ may be related to changes in passive or active transport of the peptide through the endothelium, we examined changes in BBB junctional proteins and LRP-1. Similar to our previous findings in a repeated low-intensity blast model (Heyburn et al., 2021), there was dysregulation of the endothelial proteins occludin, claudin 5, and ZO-1 but not claudin-5 (FIG. 28A). Occludin levels increased 33% 1 d post-blast (Kruskal-Wallis followed by Dunn, p<0.05 for 1 d blast vs sham) and ZO-1 levels decreased 24% and 20% at 1 d and 28 d post-blast, respectively (Kruskal-Wallis followed by Dunn, p<0.05 in each case). Claudin 5 showed a 15% increase at the 1 d time point (Welch's ANOVA followed by Dunnett's, p<0.05 for 1 d vs sham). Perhaps more significantly, we observed an increase in LRP-1 at the 1 d time point. To further characterize the increase in LRP-1, immunohistochemistry was used to study co-localization of LRP-1 with the vasculature (FIG. 28B). Assessment of LRP-1 immunofluorescence in the cortex increased 24 h after exposure to blast in the cortex (FIG. 8C: Kruskal-Wallis followed by Dunn, p<0.001). Notably, there was a ˜30% increase in LRP-1 co-localization with the vascular smooth muscle component smoothelin at 24 h relative to sham (FIG. 28D; Kruskal-Wallis followed by Dunn, p<0.01).

AQP4 Levels are Altered after Exposure to Blast

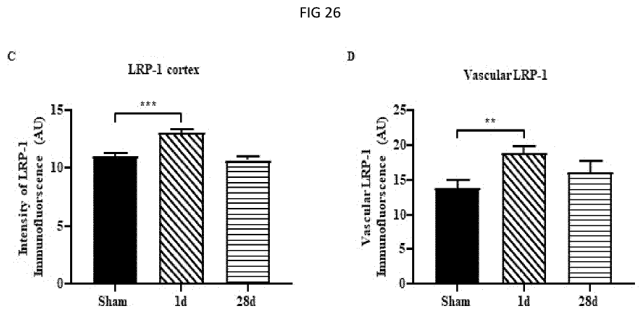

Assessment of immunofluorescence of AQP4 in the cortex showed that there was an overall 17% increase in AQP4 immunoreactivity 1 d after exposure to blast and a 19% decrease 28 d post-blast relative to sham (Welch's ANOVA W=25.97, p<0.0001 followed by Dunnett's, p<0.05 vs sham in each case; FIG. 26A). These changes in AQP4 expression did not correlate with any significant alterations in astrocytes as assessed by GFAP immunofluorescence (Kruskal-Wallis p=0.07; FIG. 268). We further investigated the expression of AQP4 isoforms in cortical tissue using western blotting (FIG. 26C and FIG. 26D). Exposure to blast at the 1 d time point was associated with increase in both the M1 (ANOVA F=5.51, p=0.15 followed by Dunnett's, p<0.05) and M23 (Welch's ANOVA W=25.5, p<0.001 followed by Dunnett's p<0.001) isoforms of AQP4. The M1-to-M23 ratio was reduced 1 d after exposure to blast but increased at 28 d after exposure (ANOVA F=20.20, p<0.0001 followed by Dunnett's p<0.01 vs sham in each case).

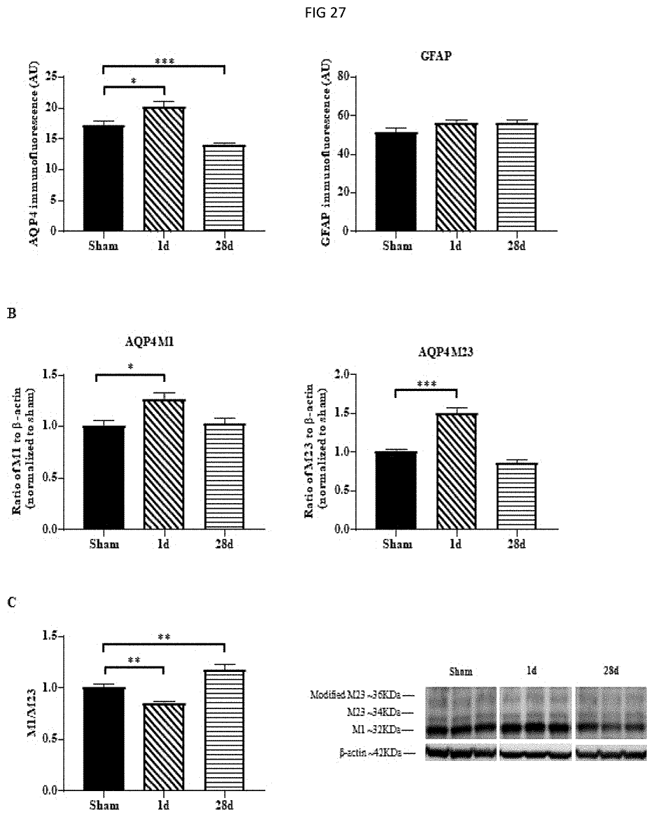

To determine the effects of blast on perivascular AQP4 levels, we determined the relative perivascular immunofluorescence of GFAP and AQP4, separately, as well as the co-localization of the two signals (FIG. 27A). Perivascular AQP4 showed a 47% decrease 1 d after exposure to blast and a 34% decrease 28 d post-blast relative to sham (Kruskal-Wallis p<0.0001 followed by Dunn's p<0.0001 vs sham in each case; FIG. 27B). We also found that there was an 18% reduction at 1 d and a ˜10% reduction at 28 d in perivascular GFAP immunofluorescence relative to sham (Kruskal-Wallis p<0.0001 followed by Dunn's, p<0.001 vs sham in each case), in spite of lack of change in overall GFAP immunoreactivity in the cortex. Interestingly, analysis of Pearson's coefficient of co-localization showed that there was an increase (˜15%) in the perivascular co-localization of AQP4 with GFAP 1 d after exposure to blast (Welch's ANOVA W=18.68, p<0.0001 followed by Dunnett's, p<0.0001). Collectively, the data demonstrate complex changes in AQP4 expression and localization, which appear to increase the polarization of AQP4 to astrocytic GFAP-labeled endfeet directly surrounding the microvessels.

### Discussion

Although some epidemiological studies found an association between TBI and the development of neurodegenerative disease later in life, the long-term effects of subclinical exposure to impact forces, primarily in contact sport, remains a topic of significant interest. Exposure to repetitive low-intensity BOP is a prevalent health concern in military and civilian law enforcement personnel with similar concerns for long-term sequalae. Subtle neurological changes may result from repetitive subclinical blast exposures which could lead to neurological dysfunction. Understanding the underlying biology of changes related to low-intensity blast exposures is important for improving brain injury risk assessment tools and exposure guidelines. Although numerous studies in humans and animal models demonstrate increase in Aβ levels hours to days after a non-blast TBI event, the relationship is less clear in blast-related TBI. The findings of this example demonstrate that exposure to low-intensity blast leads to a reduction in detergent-soluble Aβ that is related to changes in Aβ clearance mechanisms, with some possible contribution of increase in the non-amyloidogenic processing of APP.

This study extends our previous findings (De Gasperi et al., 2012; Perez Garcia et al., 2021) and demonstrates that exposure to a single low-intensity blast overpressure of 37 kPa (or 5.4 psi) is associated with reduction in detergent-soluble Aβ monomers and an increase in PBS-soluble Aβ40 at the 1 d post-blast time point. Although Aβ dynamics in the brain are complex, elevation in brain levels of water/PBS soluble and detergent/Triton-soluble forms of Aβ have been described in AD and are associated with synaptic loss and dementia. Aβ40 and 42 also decreased in plasma acutely but increased 28 d post-blast. The correlation between plasma levels of Aβ and amyloid burden in the brain remains unclear. However, some studies reported reduction in plasma levels of Aβ peptides in AD dementia patients and a correlation of the lower levels of plasma Aβ with increased levels in neocortical Aβ as assessed by positron emission tomography (PET). Conversely, in human non-blast TBI, elevated levels of plasma Aβ42 have been correlated with injury severity and morbidity. Repetitive exposure to blast in military personnel has also been associated with elevated levels of serum Aβ 40 and 42 hours after the final exposure. Our findings demonstrating a reduction in brain detergent-soluble Aβ with a concomitant reduction in plasma Aβ in the acute phase after exposure to a single low-intensity blast are different from those reported in human AD, impact TBI, and with repetitive low-intensity blast exposure in operational situations.

The discrepancy between our findings and the observations in AD and non-blast TBI are most likely due to differences in the pathogenic mechanisms of these insults. The difference between our findings in this animal model and the findings in blast-exposed services members may be due to differences in sampling time after blast exposure or due to difference in the intensity and other parameters of the blast exposure experienced by service members. Of note, the significant increase in plasma Aβ 42 levels in our model 28 d post-blast may be an indication of vascular injury, as elevation of serum levels of Aβ have been reported in patients with cerebral microbleeds, hypertension and other vascular diseases. This conclusion is also supported by our work in experimental animal models which demonstrate that injury to the cerebral vasculature is a prominent and prolonged effect of exposure to blast overpressure (Gama Sosa et al., 2014; Abutarboush et al., 2019; Gama Sosa et al., 2019; Gama Sosa et al., 2021; Kawoos et al., 2021b). The reduction in plasma levels of Aβ may involve more complex dynamics some of which may involve alterations in both production and clearance of Aβ and are discussed further below.

Our study addressed mechanisms that may have contributed to the observed changes in Aβ levels. The findings we report indicate that the reduction in detergent-soluble Aβ in the brain in the acute phase cannot be attributed to oligomerization of the monomeric protein into larger species. Aβ 42 has a higher aggregation rate than Aβ 40 and is more likely to form the highly neurotoxic oligomers. It is noteworthy that PBS-soluble Aβ 40 and 42 and Triton-soluble Aβ 42 in blast-exposed animals were slightly elevated compared to their respective sham groups at 28 d. However, the elevation in oligomers at 28 d was not different between sham and blast-exposed animals. The increase in soluble Aβ oligomers over time (1 d vs. 28 d) was independent of blast exposure and may be an effect of increase in the animals' age. Evidence suggests that aging neurons are more susceptible to the neurodegenerative changes induced by Aβ. These observations should be investigated further in future work.

In a series of studies, we also assessed factors that may affect the production of Aβ, including the expression levels of APP and the secretases involved in the amyloidogenic cleavage of APP, BACE1, and PSN1. Elevated APP levels have been reported in human and animal studies following non-blast TBI and may be due to increased expression or accumulation of the protein due to impaired axonal transport along injured axons (Pierce et al., 1996; Olsson et al., 2004; Tsitsopoulos and Marklund, 2013). APP levels did not change in the Triton fraction 1 d post-blast. There was, however, a reduction in the APP Triton fraction at 28 d does not correspond with the observations of Aβ levels at 28 d. Similarly, the observed reductions in the levels of APP in PBS fractions do not correspond with the observed changes in PBS or Triton-soluble pools of Aβ in the brain. It is unknown at this time what the PBS versus Triton-soluble APP fractions represent and how each of the two pools contributes to Aβ levels. Unlike the findings of this study, our previous work showed that Triton APP levels increased after exposure to low-intensity blast with no APP staining in axons of blast-exposed animals (De Gasperi et al., 2012). The differences may be related to differences in the antibodies used for the study or the semi-quantitative analysis of APP bands on the immunoblots.

We did not observe significant changes in BACE1 and PSN1 after blast exposure. These results are similar to our previous findings. This report extends our previous research and demonstrates that the statistically non-significant reduction in the levels of BACE11 d after exposure to blast correlated with a significant reduction in the enzymatic activity of BACE1. This change could lead to a reduction in the production of Aβ from APP, which could partially account for some of the observed reduction in Aβ.

Assessment of the effects of low-intensity blast exposure on the α-secretases, ADAM10, and ADAM17 revealed unexpected findings. Although ADAM10 is most likely the main constitutive α-secretase in neurons, both ADAM proteins cleave APP via a non-amyloidogenic pathway releasing sAPPα, the N-terminal domain of APP, which has neuroprotective properties. In fact, overexpression of ADAM10 has been associated with reduction in Aβ plaque loads in AD models. Additionally, reduction in ADAM10 has been documented in AD patients and may be associated with the increase in Aβ load and neurodegeneration. Similarly, ADAM17 (or TACE) has been shown to affect processes important for neuronal proliferation. ADAM17 is involved in the shedding of over 80 cytokines and is involved in the progression of CNS pathologies including multiple sclerosis and TBI due to its role in neuroinflammation. Although ADAM17 is less studied than ADAM10 in relation to neurodegenerative disorders, it has been shown that ADAM17 co-localizes with amyloid plaques in AD brains, suggesting a possible connection to inflammatory or APP-processing occurring at those sites. In addition, reduction in ADAM17 activity has been associated with decreased accumulation of Aβ and reduction in TNF-α.

The reduction in ADAM17 levels observed in this study may result in changes in the signaling of certain cytokines that are substrates of the ADAM17 proteolytic activity, including TNF-α and the IL-6 receptor and, hence, IL-6 signaling pathways. Our observations show that the reduction in ADAM17 at the 1 d time point post-blast correlated with a minor decrease in TNF-α in the brain and a robust increase in plasma levels of TNF-α. Plasma levels of TNF-α nearly tripled compared to sham values 1 d after exposure to a single low-level blast and returned to near sham values by 28 d post-blast. In the CNS, TNF-α is produced by microglia and neurons, but can be produced by other cells upon injury. Similar to other cytokines, TNF-α can move freely across the intact BBB into the blood compartment and, conversely, peripheral blood-borne immunocompetent TNF-α-producing cells can also enter the brain and contribute to neuroinflammation. Therefore, it is not possible to determine whether the TNF-α detected in plasma is peripheral or central in origin without further analyses. Data generated by our group show that experienced breachers exposed to hundreds or thousands of repeated low-level blast over a career exhibit elevated serum levels of brain-derived TNF-α and IL-6 without an increase in serum cytokine levels of these factors. The time-course of this observed elevation and how it correlates with our animal study are difficult to discern. Part of the complexity in extrapolating the findings in the animal model to observations in service members lies in differences in frequency, intensity, and chronicity of blast exposures in these studies. However, these studies demonstrate an imbalance in the neuroinflammatory response following exposure to low intensity blast which could lead to long-term perturbations in these systems with cumulative exposure over a career.

In non-blast TBI, inflammation, as measured by activated microglia and macrophages, was detectable in the brain years after injury in both human cases and animal studies, and depletion of microglia has been shown to prevent TBI-induced neuropathology and behavioral impairments. Several epidemiological studies have shown the sparing of cognitive decline in Alzheimer's patients treated with anti-TNF-α agents. In addition, the risk for Alzheimer's Disease is significantly reduced in rheumatoid arthritis patients receiving anti-TNF-α drugs, such as etanercept, for treatment of rheumatoid arthritis. Collectively, these findings implicate a central role for inflammation and TNF-α in specific in the pathogenesis of neuronal and vascular derangement and subsequent cognitive and behavioral impairments induced by TBI and neurodegenerative disease. These relationships remain to be elucidated in blast-induced TBI and with subclinical exposures with the potential to contribute to cumulative neurological effects. In particular, the significance of the reduction of ADAM17 we report as it relates to TNF-α production and changes in neurodegenerative proteins including Aβ may present an approach for the treatment of certain brain diseases. Future studies focusing on investigating these relationships are indicated.

Concomitant to changes in TNF-α in our 5.4 psi single blast model were changes in vascular-related proteins, involved in removal of Aβ from the brain. Clearance of potentially toxic products of neuronal activity, including Aβ, via vascular-related mechanisms has gained much interest in recent years. At least three vascular-mediated pathways have been proposed: a transvascular pathway involving the BBB and LRP-1-mediated transcytosis; a glymphatic (paravascular) pathway involving AQP4 water channels in astrocytic endfeet; and a perivascular pathway in which solutes in the interstitium move from the brain parenchyma along the vasculature to reach the surface of the brain and eventually drain into cervical lymph nodes, possibly via dural lymph vessels. Many pressing questions exist around the detailed clearance “routes” of the perivascular and glymphatic pathways and the interaction of the two pathways and the relative contribution of each pathway to clearance of neurotoxic proteins. Our current study was not designed to address these questions and only explored the possible relationship between the observed reduction in Aβ after low-intensity blast and the changes in the better-understood transvascular, BBB-mediated pathway of Aβ clearance, as well as those in the AQP4-mediated pathway by examining the levels and localization of AQP4.

Exposure to blast is associated with dysregulation of BBB endothelial tight junctional proteins (Lucke-Wold et al., 2015; Heyburn et al., 2021; Kawoos et al., 2021a). Both increases and decreases in the levels of claudin5, occludin, and ZO-1 have been reported in the literature. In our experience, exposure to high intensity blast (˜19 psi) was associated with reduction in these junctional proteins in the acute phase post-exposure (Kawoos et al., 2021a). Unlike our findings in the high-intensity blast model, here we report an increase in occluding and claudin 5 1 d after exposure to low-intensity blast and a reduction in ZO-1 at both the 1 and 28 d time points. The increase in brain levels of LRP1 may be associated with enhanced clearance of Aβ by LRP1 transcytosis of the peptide through the endothelium into the blood. Expressed in vascular (endothelial, pericyte) and non-vascular cells in the brain (neurons, astrocytes and microlglia) (Ramanathan et al., 2015), LRP1 clearance of Aβ is a major physiological mechanism of Aβ removal from the brain that occurs at the BBB (Shibata et al., 2000). LRP1 has been shown to preferentially and rapidly bind Aβ 40 on the abluminal side of the capillary endothelium and transport the peptide across the BBB with 8-fold higher affinity than Aβ 42 (Deane et al., 2004). Additionally, reduction in the levels of LRP1 have been associated with Aβ accumulation in AD (Kang et al., 1997) and transgenic AD animal models (Deane et al., 2004). Studies have shown that ADAM10 and ADAM17 mediate LRP1 shedding into soluble LRP1 and that levels of soluble LRP1 increase significantly with age and correlate with increase in Aβ (Liu et al., 2009b). The reduction in Triton-soluble Aβ along with reduction in ADAM17 and increase in LRP1 at 24 h after exposure to blast are consistent with these studies and suggest a role for blast-induced changes in the endothelium in Aβ clearance. The changes in LRP1 we describe appear to be short-lived as the levels of LRP1 return to sham values 28 d post-blast.

Disruption in AQP4 expression has been associated with AD in humans (Pérez et al., 2007; Zeppenfeld et al., 2017) and with increase in Aβ deposits in animal models (Yang et al., 2011; Xu et al., 2015). Similar to our findings in blast-exposed rats, increase in overall cortical AQP immunoreactivity and decrease of perivascular AQP4 has been observed in AD patients (Zeppenfeld et al., 2017). Astrocytic AQP4 has been shown to play a vital role in bulk clearance of interstitial solutes, including Aβ (Iliff et al., 2012) and redistribution of AQP4 away from astrocytic perivascular endfeet has been associated Aβ deposits in AD models (Yang et al., 2011). Although brain injury has been known to alter AQP4 levels, the changes remain largely subject to debate. While some studies have shown an acute upregulation of AQP4 in response to TBI (Kapoor et al., 2013; Ren et al., 2013), other studies have demonstrated a decrease in AQP4 (L U et al., 2013). Here we show enhancement in co-localization of AQP4 with GFAP-positive astrocytic endfeet surrounding cortical microvessels 1 d after exposure to intensity blast, despite perivascular decrease of both GFAP and AQP4 post-blast. We can speculate that this finding may affect the degree Aβ clearance. Our data showing changes in the ratio of AQP4 isoforms (discussed below) also lends further support to this interpretation.

Our study also showed that exposure to a single low-intensity blast overpressure the ratio of the AQP4 M1 and M23 isoforms. The increase in M1 and M23 expression 1 d after blast may be associated with enhanced fluid movement as an acute response to blast overpressure. This could indicate increased glymphatic clearance, which may also be supported by our immunohistochemistry data, offering a possible explanation for the decreased level of detergent Aβ40 and 42 at the 1 d post-blast time point. Blast exposure was also found to significantly increase the ratio of M1:M23 with time after blast exposure. While the decrease in M23 levels 28 d post-blast was responsible for this change, it suggests a reduction in orthogonal arrays of particles (OAP) size to be a chronic response to blast. Only AQP4 is known to aggregate into higher order OAPs (Nicchia et al., 2010). The ratio of M1:M23 appears to be the major determinant of the size of in vivo OAPs, where the larger the M1:M23 ratio, the smaller the size of the OAP (Nicchia et al., 2013). With the M23 isoform having a higher single-channel water permeability coefficient compared to M1 (Silberstein et al., 2004), it is possible that exposing animals to low-intensity blast significantly compromises bulk fluid movement in the brain 28 d after blast exposure. This, however, was not supported by an increase in Aβ in our 28 days blast animals. While the redistribution of AQP4 towards astrocytic endfeet and the changes in AQP4 isoforms may partially explain the reduction in detergent levels of Aβ, they do not explain the observed elevation in PBS Aβ levels 1 d post-blast. Future work will focus on using imaging techniques to examine the effects of low-intensity blast exposure on AQP4-mediated clearance and the seemingly time-dependent response to blast exposure.

Our data demonstrate changes in neurodegenerative proteins and specifically Aβ following exposure to low-intensity sub-concussive blast are to be considered alongside with other factors, including changes in both central and peripheral inflammation. How much of the observed alterations in Aβ can be attributed to changes in APP-cleaving secretases, trans-endothelial clearance via LRP1, or paravascular glymphatic AQP4-mediated clearance cannot be determined by this initial investigation. Factors including enzymatic expression and activity, molecular size, arterial pulsation, and AQP4 localization (Tarasoff-Conway et al., 2015) affect the various β-clearing mechanisms explored in this study. The long-term effects of the blast-induced observed changes in Aβ remain to be explored. Given the observed vascular or perivascular protein changes in this low-intensity blast model, future work will focus on the cerebrovascular outcomes of these low-intensity exposure levels.

### REFERENCES

1. Kawarabayashi, T., Younkin, L. H., Saido, T. C., Shoji, M., Ashe, K. H. and Younkin, S. G. (2001). Age-dependent changes in brain, CSF, and plasma amyloid (beta) protein in the Tg2576 transgenic mouse model of Alzheimer's disease. J Neurosci 21, 372-381.
2. Steele, J. W., Kim, S. H., Cirrito, J. R., Verges, D. K., Restivo, J. L., Westaway, D., Fraser, P., Hyslop, P. S., Sano, M., Bezprozvanny, I., Ehrlich, M. E., Holtzman, D. M. and Gandy, S. (2009). Acute dosing of latrepirdine (Dimebon), a possible Alzheimer therapeutic, elevates extracellular amyloid-beta levels in vitro and in vivo. Mol Neurodegener 4, 51.
3. Perez Garcia G, De Gasperi R, Tschiffely A E, Gama Sosa M A, Abutarboush R, Kawoos U, Statz J K, Ciarlone S, Reed E, Jeyarajah T, Perez G M, Otero-Pagan A, Pryor D, Hof P R, Cook D G, Gandy S, Elder G A, Ahlers S T (2021) Repetitive Low-Level Blast Exposure Improves Behavioral Deficits and Chronically Lowers Aβ42 in an Alzheimer Disease Transgenic Mouse Model. J Neurotrauma 38:3146-3173.
4. De Gasperi R, Gama Sosa M A, Kim S H, Steele J W, Shaughness M C, Maudlin-Jeronimo E, Hall A A, Dekosky S T, McCarron R M, Nambiar M P, Gandy S, Ahlers S T, Elder G A (2012) Acute blast injury reduces brain abeta in two rodent species. Front Neurol 3:177.
5. Harper, M. M., Hedberg-Buenz, A., Herlein, J., Abrahamson, E. E., Anderson, M. G., Kuehn, M. H., Kardon, R. H., Poolman, P. and Ikonomovic, M. D. (2019). Blast-Mediated Traumatic Brain Injury Exacerbates Retinal Damage and Amyloidosis in the APPswePSENd19e Mouse Model of Alzheimer's Disease. Invest Ophthalmol Vis Sci 60, 2716-2725.
6. Maigler, K. C., Buhr, T. J., Park, C. S., Miller, S. A., Kozlowski, D. A. and Marr, R. A. (2021). Assessment of the Effects of Altered Amyloid-Beta Clearance on Behavior following Repeat Closed-Head Brain Injury in Amyloid-Beta Precursor Protein Humanized Mice. J Neurotrauma.
7. Gama Sosa M A, De Gasperi R, Janssen P L, Yuk F J, Anazodo P C, Pricop P E, Paulino A J, Wicinski B, Shaughness M C, Maudlin-Jeronimo E, Hall A A, Dickstein D L, McCarron R M, Chavko M, Hof P R, Ahlers S T, Elder G A (2014) Selective vulnerability of the cerebral vasculature to blast injury in a rat model of mild traumatic brain injury. Acta Neuropathol Commun 2:67-67.
8. Gama Sosa M A, De Gasperi R, Pryor D, Perez Garcia G S, Perez G M, Abutarboush R, Kawoos U, Hogg S, Ache B, Janssen W G, Sowa A, Tetreault T, Cook D G, Tappan S J, Gandy S, Hof P R, Ahlers S T, Elder G A (2021) Low-level blast exposure induces chronic vascular remodeling, perivascular astrocytic degeneration and vascular-associated neuroinflammation. Acta Neuropathol Commun 9:167.
9. Gama Sosa M A, De Gasperi R, Perez Garcia G S, Perez G M, Searcy C, Vargas D, Spencer A, Janssen P L, Tschiffely A E, McCarron R M, Ache B, Manoharan R, Janssen W G, Tappan S J, Hanson R W, Gandy S, Hof P R, Ahlers S T, Elder G A (2019) Low-level blast exposure disrupts gliovascular and neurovascular connections and induces a chronic vascular pathology in rat brain. Acta Neuropathol Commun 7:6.
10. Kawoos U, Abutarboush R, Reed E, Zhang R, Patterson J, Watson C, Gu M, Jeyarajah T, Ahlers S T (2021b) Temporal changes in cerebral blood flow and vascular reactivity in a rat model of blast traumatic brain injury. In: National Neurotrauma Virtual Symposium. Virtual.
11. Abutarboush R, Gu M, Kawoos U, Mullah S H, Chen Y, Goodrich S Y, Lashof-Sullivan M, McCarron R M, Statz 1K, Bell R S, Stone J R, Ahiers S (2019) Exposure to Blast Overpressure Impairs Cerebral Microvascular Responses and Alters Vascular and Astrocytic Structure. J Neurotrauma.

What is claimed is:

1) A method for treating and/or preventing Alzheimer's disease or other neurodegenerative diseases in a subject, comprising
a) identifying a subject at risk of or in early stage of Alzheimer's disease or other neurodegenerative diseases; and
b) exposing said subject to repetitive low intensity blast overpressure.

2) The method of claim 1, wherein said method treats and/or prevents Alzheimer's disease or other neurodegenerative diseases in said subject by
a) improving behavioral deficits;
b) reducing abnormal brain accumulation or deposition of beta-amyloid;
c) reducing brain inflammation; or
d) a combination thereof

3) The method of claim 1, wherein said low intensity blast overpressure is at subclinical blast level.

4) The method of claim 1, wherein said subclinical blast overpressure is approximately equal or less than 10 psi.

5) The method of claim 1, wherein said subject is exposed to repetitive low intensity blast overpressure intermittently for a prolonged period.

6) The method of claim 2, wherein said behavioral deficits include anxiety, impaired cognition, social interactions, loss of spatial, impairment in short term memory, speech impediment, visuospatial skills impairment, orientation impairment, and difficulty in reasoning or problem-solving, difficulty in handling complex tasks, difficulty in concentrating, planning and organizing or a combination thereof.

7) The method of claim 2, wherein said reduction of abnormal brain accumulation or deposition of beta-amyloid is achieved by
a) Decreasing APP-cleaving secretases;
b) Increasing trans-endothelial clearance via LRP1;
c) Improving paravascular glymphatic AQP4-mediated clearance; or
d) A combination thereof.

8) The method of claim 1, further comprises at least once administering to said subject an effective amount of one or more therapeutic agent or therapy.

9) The method of claim 7, wherein said therapeutic agent
i) prevents β-amyloid deposition;
ii) reduces β-amyloid production;
iii) improves β-amyloid clearance;
iv) improves brain inflammation;
v) inhibits of BACE1; or
vi) a combination thereof

10) A method to reduce abnormal accumulation of brain proteins in a subject at risk or in early stage of Alzheimer's disease or other neurodegenerative diseases, comprising
a) Identifying a subject at risk of developing Alzheimer's disease or other neurodegenerative diseases caused by abnormal accumulation of proteins in the brain; and
b) Exposing said subject to repetitive low intensity blast overpressure.

11) The method of claim 10, wherein said proteins include α-synuclein, tau, amyloid precursor protein (APP), amyloid β protein (Aβ) or a combination thereof.

12) The method of claim 11, wherein said amyloid β protein (Aβ) is Aβ 42.

13) The method of claim 10, wherein said low intensity blast overpressure is at subclinical blast level.

14) The method of claim 13, wherein said subclinical blast overpressure is equal or less than 10 psi.

15) A method to improve brain inflammation in patients at risk for Alzheimer's disease or other neurodegenerative diseases, comprising
a) Identifying a patient at risk of developing Alzheimer's disease or other neurodegenerative diseases caused by abnormal accumulation of proteins in the brain; and
b) Exposing said subject to repetitive low intensity blast overpressure.

16) The method of claim 15, wherein said improvement of brain inflammation include reduction in brain ADAM17, and increase in serum levels of brain-derived TNF-α and IL-6.

17) A device or system treating and/or preventing Alzheimer's disease or other neurodegenerative diseases in a subject, wherein said device is capable of safely deliver pulsed pressure wave to a subject.

18) The device of claim 17, wherein said pulsed pressure wave is delivered to said subject at subclinical blast level.

19) The device of claim 18, wherein said pulsed pressure wave is delivered to said subject at approximately equal or less than 10 psi.
